(19) United States
(12) Patent Application Publication
(10) Pub. No.: US 2008/0118923 A1
(43) Pub. Date: May 22, 2008

(54) METHOD AND COMPOSITIONS FOR DISCRIMINATING GRAM-NEGATIVE AND GRAM-POSITIVE BACTERIA

(75) Inventors: Kyung-hee PARK, Yongin-si (KR); Jung-nam LEE, Yongin-si (KR); Sook-young KIM, Yongin-si (KR)

Correspondence Address: CANTOR COLBURN, LLP, 20 Church Street, 22nd Floor, Hartford, CT, 06103, US

(73) Assignee: SAMSUNG ELECTRONICS CO., LTD., Suwon-si (KR)

(21) Appl. No.: 11/739,437

(22) Filed: Apr. 24, 2007

(30) Foreign Application Priority Data
Sep. 28, 2006 (KR) 10-2006-0095037

(51) Int. Cl. C12Q 1/68; C07H 21/00

(52) U.S. Cl. 435/6; 536/24.32; 536/24.33

(57) ABSTRACT

Provided are an oligonucleotide primer set for amplifying at least one target sequence of the 16S rRNA gene in gram-negative bacteria and at least one target sequence of the 16S rRNA gene in gram-positive bacteria, a probe set specifically hybridizing with at least one target sequence of the 16S rRNA gene in gram-negative bacteria and at least one target sequence of the 16S rRNA gene in gram-positive bacteria, a microarray comprising the probe set, a method of discriminating gram-negative bacteria and gram-positive bacteria, and a kit for discriminating gram-negative bacteria and gram-positive bacteria, including the primer set.

This application claims priority from Korean Patent Application No. 10-2006-0095037, filed on Sep. 28, 2006, and all the benefits accruing therefrom under 35 U.S.C. §119, the disclosure of which is incorporated herein in its entirety by reference.

### BACKGROUND OF THE INVENTION

1. Field of the Invention

The present invention relates to a method and compositions for discriminating gram-negative and gram-positive bacteria. More particularly, the present invention relates to an oligonucleotide primer set for amplifying at least one target sequence of a 16S rRNA gene in gram-negative bacteria and at least one target sequence of a 16S rRNA gene in gram-positive bacteria, a probe set specifically hybridizing with at least one target sequence of a 16S rRNA gene in gram-negative bacteria and at least one target sequence of a 16S rRNA gene gram-positive bacteria, a microarray comprising the probe set, methods of discriminating gram-negative bacteria and gram-positive bacteria, and a kit for discriminating gram-negative bacteria and gram-positive bacteria, including the primer set.

2. Description of the Related Art

The detection and accurate identification of bacteria play an important role in clinical diagnostics. As an example, a specific pathogen in a bacterial infection must be identified as quickly as possible in order to initiate appropriate treatment.

In routine medical diagnostics, bacteria have traditionally been identified via selective media and subsequent investigation of the biochemical properties. Frequently, however, it is impossible to accurately identify the species of bacteria. Moreover, these studies are time-consuming, and require growing a pure bacterial culture for more extensive studies. Various groups of bacteria with various culture conditions are difficult to access by traditional culture methods.

In recent years, nucleic acid-based methods to detect bacteria have been introduced. These methods include, for example, carrying out nucleic acid amplification reactions using species-specific primers. Detection is generally achieved through gel electrophoresis or hybridizing with probes immobilized on microtiter plates. However, such techniques are not suitable for detecting multiple pathogenic microorganisms simultaneously. One approach to the problem is to perform nucleic acid amplification using a mixture of different species-specific amplification primers and corresponding probes.

U.S. Patent Publication No. 2004/0171007 discloses a method of detecting gram-positive bacteria and primers used for the method. U.S. Patent Publication No. 2002/0081606 discloses a method of detecting and identifying gram-positive bacteria in a sample and primers used for the method.

In spite of the above-described conventional techniques, no primer set capable of discriminating 100 or more gram-negative and gram-positive bacterial species has been disclosed. Furthermore, no probe capable of discriminating 100 or more gram-negative and gram-positive bacterial species has been disclosed.

### SUMMARY OF THE INVENTION

The present invention provides a primer set capable of discriminating gram-negative bacteria and gram-positive bacteria.

In one embodiment, an oligonucleotide primer set comprises: a first oligonucleotide set comprising an oligonucleotide set comprising an oligonucleotide consisting of at least 10 contiguous nucleotides of SEQ ID NO: 1 and an oligonucleotide consisting of at least 10 contiguous nucleotides of SEQ ID NO: 2; an oligonucleotide set comprising an oligonucleotide consisting of at least 10 contiguous nucleotides of SEQ ID NO: 3 and at least one oligonucleotide consisting of at least 10 contiguous nucleotides of SEQ ID NO: 4; or an oligonucleotide set comprising at least one oligonucleotide consisting of at least 10 contiguous nucleotides of SEQ ID NO: 5 and at least one oligonucleotide consisting of at least 10 contiguous nucleotides of SEQ ID NO: 6; and a second oligonucleotide set comprising an oligonucleotide set comprising at least one oligonucleotide consisting of at least 10 contiguous nucleotides of SEQ ID NO: 7 and at least one oligonucleotide consisting of at least 10 contiguous nucleotides of SEQ ID NO: 8; an oligonucleotide set comprising at least one oligonucleotide consisting of at least 10 contiguous nucleotides of SEQ ID NO: 9 and at least one oligonucleotide consisting of at least 10 contiguous nucleotides of SEQ ID NO: 10; or an oligonucleotide set comprising at least one oligonucleotide consisting of of at least 10 contiguous nucleotides of SEQ ID NO: 11 and at least one oligonucleotide consisting of at least 10 contiguous nucleotides of SEQ ID NO: 12, wherein the oligonucleotide primer set amplifies a target sequence of a 16S rRNA gene in gram-negative bacteria and a target sequence of a 16S rRNA gene in gram-positive bacteria.

The present invention also provides a probe set capable of discriminating gram-negative bacteria and gram-positive bacteria.

In an embodiment, an oligonucleotide probe set comprises a first oligonucleotide probe selected from the group consisting of an oligonucleotide probe capable of hybridizing with a nucleotide region of a 16S rRNA gene of gram-negative bacteria corresponding to positions 358-579 of SEQ ID NO 31, wherein the oligonucleotide probe consists of at least 10 contiguous nucleotides of a sequence selected from the group consisting of SEQ ID NOS: 13-15 and a complement of SEQ ID NOS: 13-15; an oligonucleotide probe capable of hybridizing with a nucleotide region of a 16S rRNA gene of gram-negative bacteria corresponding to positions 823-1240 of SEQ ID NO 32, wherein the oligonucleotide probe consists of at least 10 contiguous nucleotides of a sequence selected from the group consisting of SEQ ID NOS: 16-18 and a complement of SEQ ID NOS: 16-18; and an oligonucleotide probe capable of hybridizing with a nucleotide region of a 16S rRNA gene of gram-negative bacteria corresponding to positions 290-595 of SEQ ID NO 33, wherein the oligonucleotide probe consists of at least 10 contiguous nucleotides of a sequence selected from the group consisting of SEQ ID NOS: 19-21 and a complement of SEQ ID NOS: 19-21; and a second oligonucleotide probe selected from the group consisting of an oligonucleotide probe capable of hybridizing with a nucleotide region of a 16S rRNA gene of gram-positive bacteria corresponding to positions 885-1169 of SEQ ID NO 34, wherein the oligonucleotide probe consists of at least 10 contiguous nucleotides of a sequence selected from the group consisting of SEQ ID NOS: 22-24 and a complement of SEQ ID NOS: 22-24; an oligonucleotide probe capable of hybridizing with a nucleotide region of a 16S rRNA gene of gram-positive bacteria corresponding to positions 30-150 of SEQ ID NO 35, wherein the oligonucleotide probe consists of at least 10 contiguous nucleotides of a sequence selected from the group consisting of SEQ ID NOS: 25-27 or a complement of SEQ ID NOS: 25-27; and an oligonucleotide probe capable of hybridizing with a nucleotide region of a 16S rRNA gene of gram-positive bacteria corresponding to positions 594-749 of SEQ ID NO 36, wherein the oligonucleotide probe consists of at least 10 contiguous nucleotides of a sequence selected from the group consisting of SEQ ID NOS: 28-30 or a complement of SEQ ID NOS: 28-30.

The present invention also provides a microarray comprising the probe set.

The present invention also provides methods of discriminating gram-negative bacteria and gram-positive bacteria in a sample.

In an embodiment, the method comprises using genomic DNA in a sample as a template and a first oligonucleotide primer set specific for amplifying a target region specific to a gram-negative bacterium in a 16S rRNA gene and a second oligonucleotide primer set specific for amplifying a target region specific to a gram-positive bacterium in a 16S rRNA gene in an amplification reaction; and detecting the gram-negative bacterium in the sample when the gram-negative bacterium-specific target sequence is amplified and detecting the gram-positive bacterium in the sample when the gram-positive bacterium-specific target sequence is amplified.

In an embodiment, the method comprises contacting a sample with the oligonucleotide probe set to allow hybridization between a target sequence of the sample and the first and second oligonucleotide probes; and detecting hybridization between the first and second oligonucleotide probes and the target sequence of the sample to discriminate gram-negative bacteria from gram-positive bacteria in the sample.

The present invention also provides a kit for discriminating gram-negative bacteria and gram-positive bacteria, including the primer set.

### BRIEF DESCRIPTION OF THE DRAWINGS

The above and other features and advantages of the present invention will become more apparent by describing in detail exemplary embodiments thereof with reference to the attached drawings in which:

FIG. 1 shows agarose gel electrophoretic results of PCR products obtained by single PCR and multiplex PCR using six primer sets according to an embodiment of the present invention.

### DETAILED DESCRIPTION OF THE INVENTION

The present invention provides an oligonucleotide primer set for amplifying at least one target sequence of a 16S rRNA gene in gram-negative bacteria and at least one target sequence of a 16S rRNA gene in gram-positive bacteria. The oligonucleotide primer set comprises: a first oligonucleotide set comprising an oligonucleotide set comprising an oligonucleotide consisting of at least 10 contiguous nucleotides of SEQ ID NO: 1 and an oligonucleotide consisting of at least 10 contiguous nucleotides of SEQ ID NO: 2; an oligonucleotide set comprising an oligonucleotide consisting of at least 10 contiguous nucleotides of SEQ ID NO: 3 and at least one oligonucleotide consisting of at least 10 contiguous nucleotides of SEQ ID NO: 4; or an oligonucleotide set comprising at least one oligonucleotide consisting of at least 10 contiguous nucleotides of SEQ ID NO: 5 and at least one oligonucleotide consisting of at least 10 contiguous nucleotides of SEQ ID NO: 6; and a second oligonucleotide set comprising an oligonucleotide set comprising at least one oligonucleotide consisting of at least 10 contiguous nucleotides of SEQ ID NO: 7 and at least one oligonucleotide consisting of at least 10 contiguous nucleotides of SEQ ID NO: 8; an oligonucleotide set comprising at least one oligonucleotide consisting of at least 10 contiguous nucleotides of SEQ ID NO: 9 and at least one oligonucleotide consisting of at least 10 contiguous nucleotides of SEQ ID NO: 10; or an oligonucleotide set comprising at least one oligonucleotide consisting of of at least 10 contiguous nucleotides of SEQ ID NO: 11 and at least one oligonucleotide consisting of at least 10 contiguous nucleotides of SEQ ID NO: 12, wherein the oligonucleotide primer set amplifies a target sequence of a 16S rRNA gene in gram-negative bacteria and a target sequence of a 16S rRNA gene in gram-positive bacteria.

For the primer set of the present invention, the target sequence of a 16S rRNA gene of a gram-negative bacteria amplified by the primer set can be at least one of the nucleotide regions corresponding to nucleotides 358-579 of SEQ ID NO 31, 823-1240 of SEQ ID NO 32, and 290-595 of SEQ ID NO 33, and the target sequence of a 16S rRNA gene of a gram-positive bacteria amplified can be at least one of the nucleotide regions corresponding to nucleotides 885-1169 of SEQ ID NO 34, 30-150 of SEQ ID NO 35, and 594-749 of SEQ ID NO 36.

The primer set of the present invention can include an oligonucleotide set for amplifying a nucleotide region of a 16S rRNA gene of a gram-negative bacteria corresponding to positions 358-579 of SEQ ID NO 31, the oligonucleotide set including an oligonucleotide consisting of at least 10 contiguous nucleotides of SEQ ID NO: 1 and an oligonucleotide consisting of at least 10 contiguous nucleotides of SEQ ID NO: 2.

The primer set of the present invention can include an oligonucleotide set for amplifying a nucleotide region of a 16S rRNA gene of a gram-negative bacteria corresponding to nucleotides 823-1240 of SEQ ID NO 32, the oligonucleotide set including an oligonucleotide consisting of at least 10 contiguous nucleotides of SEQ ID NO: 3 and an oligonucleotide consisting of at least 10 contiguous nucleotides of SEQ ID NO: 4.

The primer set of the present invention can include an oligonucleotide set for amplifying a nucleotide region of a 16S rRNA gene of a gram-negative bacteria corresponding to nucleotides 290-595 of SEQ ID NO 33, the oligonucleotide set including an oligonucleotide consisting of at least 10 contiguous nucleotides of SEQ ID NO: 5 and an oligonucleotide consisting of at least 10 contiguous nucleotides of SEQ ID NO: 6.

The primer set of the present invention can include an oligonucleotide set for amplifying a nucleotide region of a 16S rRNA gene of a gram-positive bacteria corresponding to nucleotides 885-1169 of SEQ ID NO 34, the oligonucleotide set including an oligonucleotide consisting of at least 10 contiguous nucleotides of SEQ ID NO: 7 and an oligonucleotide consisting of at least 10 contiguous nucleotides of SEQ ID NO: 8.

The primer set of the present invention can include an oligonucleotide set for amplifying a nucleotide region of a 16S rRNA gene of gram-positive bacteria corresponding to nucleotides 30-150 of SEQ ID NO 35, the oligonucleotide set including an oligonucleotide consisting of at least 10 contiguous nucleotides of SEQ ID NO: 9 and an oligonucleotide consisting of at least 10 contiguous nucleotides of SEQ ID NO: 10.

The primer set of the present invention can include an oligonucleotide set for amplifying a nucleotide region of a 16S rRNA gene of a gram-positive bacteria corresponding to nucleotides 594-749 of SEQ ID NO 36, the oligonucleotide set including an oligonucleotide consisting of at least 10 contiguous nucleotides of SEQ ID NO: 11 and an oligonucleotide consisting of at least 10 contiguous nucleotides of SEQ ID NO: 12.

The primer set of the present invention can be an oligonucleotide primer set for amplifying at least one target sequence selected from the group consisting of nucleotide regions of a 16S rRNA gene of gram-negative bacteria corresponding to positions 358-579 of SEQ ID NO. 31, 823-1240 of SEQ ID NO. 32, and 290-595 of SEQ ID NO. 33 and at least one target sequence selected from the group consisting of nucleotide regions of a 16S rRNA gene of gram-positive bacteria corresponding to positions 885-1169 of SEQ ID NO. 34, 30-150 of SEQ ID NO. 35, and 594-749 of SEQ ID NO. 36, the oligonucleotide primer set comprising: a first oligonucleotide set selected from the group consisting of the oligonucleotide set comprising an oligonucleotide consisting of the nucleotide sequence as set forth in SEQ ID NO: 1 and an oligonucleotide consisting of the nucleotide sequence as set forth in SEQ ID NO: 2; the oligonucleotide set comprising an oligonucleotide consisting of the nucleotide sequence as set forth in SEQ ID NO: 3 and an oligonucleotide consisting of the nucleotide sequence as set forth in SEQ ID NO: 4; and the oligonucleotide set comprising an oligonucleotide consisting of the nucleotide sequence as set forth in SEQ ID NO: 5 and an oligonucleotide consisting of the nucleotide sequence as set forth in SEQ ID NO: 6; and a second oligonucleotide set selected from the group consisting of the oligonucleotide set comprising an oligonucleotide consisting of the nucleotide sequence as set forth in SEQ ID NO: 7 and an oligonucleotide consisting of the nucleotide sequence as set forth in SEQ ID NO: 8; the oligonucleotide set comprising an oligonucleotide consisting of the nucleotide sequence as set forth in SEQ ID NO: 9 and an oligonucleotide consisting of the nucleotide sequence as set forth in SEQ ID NO: 10; and the oligonucleotide set comprising an oligonucleotide consisting of the nucleotide sequence as set forth in SEQ ID NO: 11 and an oligonucleotide consisting of the nucleotide sequence as set forth in SEQ ID NO: 12.

The primer set of the present invention can be an oligonucleotide primer set for amplifying nucleotide regions of a 16S rRNA gene of gram-negative bacteria corresponding to positions 358-579 of SEQ ID NO. 31, 823-1240 of SEQ ID NO. 32, and 290-595 of SEQ ID NO. 33, and nucleotide regions of a 16S rRNA gene of gram-positive bacteria corresponding to positions 885-1169 of SEQ ID NO. 34, 30-150 of SEQ ID NO. 35, and 594-749 of SEQ ID NO. 36, which comprises an oligonucleotide set including an oligonucleotide consisting of the nucleotide sequence as set forth in SEQ ID NO: 1 and an oligonucleotide consisting of the nucleotide sequence as set forth in SEQ ID NO: 2; an oligonucleotide set including an oligonucleotide consisting of the nucleotide sequence as set forth in SEQ ID NO: 3 and an oligonucleotide consisting of the nucleotide sequence as set forth in SEQ ID NO: 4; an oligonucleotide set including an oligonucleotide consisting of the nucleotide sequence as set forth in SEQ ID NO: 5 and an oligonucleotide consisting of the nucleotide sequence as set forth in SEQ ID NO: 6; an oligonucleotide set including an oligonucleotide consisting of the nucleotide sequence as set forth in SEQ ID NO: 7 and an oligonucleotide consisting of the nucleotide sequence as set forth in SEQ ID NO: 8; an oligonucleotide set including an oligonucleotide consisting of the nucleotide sequence as set forth in SEQ ID NO: 9 and an oligonucleotide consisting of the nucleotide sequence as set forth in SEQ ID NO: 10; and an oligonucleotide set including an oligonucleotide consisting of the nucleotide sequence as set forth in SEQ ID NO: 11 and an oligonucleotide consisting of the nucleotide sequence as set forth in SEQ ID NO: 12.

As used herein, the term “primer” refers to a single-stranded oligonucleotide sequence complementary to a nucleic acid sequence sought to be copied and serves as a starting point for the synthesis of a primer extension product. The length and sequence of a primer are determined such that the primer is suitable for initiating the synthesis of an extension product. Preferably, a primer is about 5-50 nucleotides in length. The length and sequence of a primer can be appropriately determined based on the complexity of the target DNA or RNA and the conditions under which the primer will be used, e.g., temperature and ionic strength.

According to the present invention, it is possible to discriminate and detect at least 69 gram-negative bacterial species and 55 gram-positive bacterial species, on the basis of their 16S rRNA gene using a small number of primer sets. The 124 bacterial species used in the present invention are summarized in Table 1 below.

In order to discriminate and detect the gram-negative bacteria and the gram-positive bacterial species in the 124 bacterial species, only three primer sets specific to one or more of the gram-negative bacteria and three primer sets specific to one or more of the gram-positive bacteria were required. Among the primer sets designed and used herein, the primer set of SEQ ID NOS: 1 and 2 can be used for detection of 66 of the gram-negative bacterial species (designated in the group column of Table 1 as “gramn1”), the primer set of SEQ ID NOS: 3 and 4 can be used for detection of two gram-negative bacterial species (designated in the group column of Table 1 as “gramn2”), and the primer set of SEQ ID NOS: 5 and 6 can be used for detection of one gram-negative bacterial species (designated in the group column of Table 1 as “gramn3”). Similarly for the 55 gram-positive bacterial species for which species-specific primers were designed, the primer set of SEQ ID NOS: 7 and 8 can be used for detection of 51 gram-positive bacterial species (designated in the group column of Table 1 as “gramp1”), the primer set of SEQ ID NOS: 9 and 10 can be used for detection of three gram-positive bacterial species (designated in the group column of Table 1 as “gramp2”), and the primer set of SEQ ID NOS: 11 and 12 can be used for detection of one gram-positive bacterial species (designated in the group column of Table 1 as “gramp3”).

Reference sequences for the 16S rRNA genes of the various groups of gram-negative and gram-positive bacterial species determined herein are SEQ ID NOS 31-36. Specifically, SEQ ID NO 31 is the consensus sequence resulting from the alignment of the 16S rRNA genes for the gram-negative bacterial sequences designated gramn1 in Table 1, SEQ ID NO 32 is the consensus sequence resulting from the alignment of the 16S rRNA genes for the gram-negative bacterial sequences designated gramn2 in Table 1, and SEQ ID NO 33 is the consensus sequence resulting from the alignment of the 16S rRNA genes for the gram-negative bacterial sequences designated gramn3 in Table 1. Specifically, SEQ ID NO 34 is the consensus sequence resulting from the alignment of the 16S rRNA genes for the gram-positive bacterial sequences designated gramp1 in Table 1, SEQ ID NO 35 is the consensus sequence resulting from the alignment of the 16S rRNA genes for the gram-positive bacterial sequences designated gramp2 in Table 1, and SEQ ID NO 36 is the consensus sequence resulting from the alignment of the 16S rRNA genes for the gram-positive bacterial sequences designated gramp3 in Table 1.

A gram-negative bacterial species other than those used to determine the 3 gram-negative bacterial groups can be classified as belonging to one of the 3 determined gram-negative bacterial groups based on comparison of its 16S rRNA gene with the three consensus sequences and placing it in the group to which it has the highest degree of homology. Similarly, a gram-positive bacterial species other than those used to determine the 3 gram-positive bacterial groups can be classified as belonging to one of the 3 determined groups based on comparison of its 16S rRNA gene with the three gram-positive bacterial group consensus sequences and placing it in the group to which it has the highest degree of homology. Thus, the primers and probes disclosed herein can be used to discriminate and detect more gram-negative bacterial species and gram-positive bacterial species than those disclosed in Table 1.

TABLE 1
Bacterial species used in the present invention

| # | full name | genus | gram-staining | group |
|---|---|---|---|---|
| 001 | Acinetobacter baumannii | Acinetobacter | Gram-negative | gramn1 |
| 002 | Acinetobacter calcoaceticus | Acinetobacter | Gram-negative | gramn1 |
| 003 | Acinetobacter lwoffi | Acinetobacter | Gram-negative | gramn1 |
| 004 | Bordetella pertussis | Bordetella | Gram-negative | gramn1 |
| 005 | Bacillus subtilis | Bacillus | Gram-positive | gramp1 |
| 006 | Bordetella ansorpii | Bordetella | Gram-negative | gramn1 |
| 007 | Bordetella avium | Bordetella | Gram-negative | gramn1 |
| 008 | Chlamydophila pneumoniae | Chlamydophila | Gram-negative | gramn3 |
| 009 | Citrobacter freundii | Citrobacter | Gram-negative | gramn1 |
| 010 | Enterobacter aerogenes | Enterobacter | Gram-negative | gramn1 |
| 011 | Enterobacter cloacae | Enterobacter | Gram-negative | gramn1 |
| 012 | Enterococcus faecalis | Enterococcus | Gram-positive | gramp1 |
| 013 | Enterococcus faecium | Enterococcus | Gram-positive | gramp1 |
| 014 | Haemophilus influenzae | Haemophilus | Gram-negative | gramn1 |
| 015 | Haemophilus aphrophilus | Haemophilus | Gram-negative | gramn1 |
| 016 | Klebsiella pneumoniae | Klebsiella | Gram-negative | gramn1 |
| 017 | Klebsiella oxytoca | Klebsiella | Gram-negative | gramn1 |
| 018 | Legionella pneumophila | Legionella | Gram-negative | gramn1 |
| 019 | Moraxella catarrhalis | Moraxella | Gram-negative | gramn1 |
| 020 | Mycoplasma pneumophila | Mycoplasma | Gram-positive | gramp3 |
| 021 | Morganella morganii | Morganella | Gram-negative | gramn1 |
| 022 | Moraxella nonliquefaciens | Moraxella | Gram-negative | gramn1 |
| 023 | Moraxella osloensis | Moraxella | Gram-negative | gramn1 |
| 024 | Pseudomonas aeruginosa | Pseudomonas | Gram-negative | gramn1 |
| 025 | Proteus mirabilis | Proteus | Gram-negative | gramn1 |
| 026 | Proteus vulgaris | Proteus | Gram-negative | gramn1 |
| 027 | Pseudomonas fluorescens | Pseudomonas | Gram-negative | gramn1 |
| 028 | Pseudomonas putida | Pseudomonas | Gram-negative | gramn1 |
| 029 | Pseudomonas stutzeri | Pseudomonas | Gram-negative | gramn1 |
| 030 | Staphylococcus aureus | Staphylococcus | Gram-positive | gramp1 |
| 031 | Streptococcus pneumoniae | Streptococcus | Gram-positive | gramp1 |
| 032 | Salmonella typhimurium | Salmonella | Gram-negative | gramn1 |
| 033 | Salmonella typhi | Salmonella | Gram-negative | gramn1 |
| 034 | Shigella flexneri | Shigella | Gram-negative | gramn1 |
| 035 | Shigella sonnei | Shigella | Gram-negative | gramn1 |
| 036 | Shigella sp | Shigella | Gram-negative | gramn1 |
| 037 | Staphylococcus cohnii | Staphylococcus | Gram-positive | gramp2 |
| 038 | Staphylococcus condimenti | Staphylococcus | Gram-positive | gramp1 |
| 039 | Staphylococcus gallinarum | Staphylococcus | Gram-positive | gramp2 |
| 040 | Staphylococcus haemolyticus | Staphylococcus | Gram-positive | gramp1 |
| 041 | Staphylococcus hominis | Staphylococcus | Gram-positive | gramp1 |
| 042 | Staphylococcus intermedius | Staphylococcus | Gram-positive | gramp2 |
| 043 | Staphylococcus lentus | Staphylococcus | Gram-positive | gramp1 |
| 044 | Staphylococcus lugdunensis | Staphylococcus | Gram-positive | gramp1 |
| 045 | Staphylococcus piscifermentans | Staphylococcus | Gram-positive | gramp1 |
| 046 | Staphylococcus xylosus | Staphylococcus | Gram-positive | gramp1 |
| 047 | Streptococcus agalactiae | Streptococcus | Gram-positive | gramp1 |
| 048 | Streptococcus bovis | Streptococcus | Gram-positive | gramp1 |
| 049 | Streptococcus canis | Streptococcus | Gram-positive | gramp1 |
| 050 | Streptococcus dysgalactiae | Streptococcus | Gram-positive | gramp1 |
| 051 | Streptococcus equi | Streptococcus | Gram-positive | gramp1 |
| 052 | Streptococcus equinus | Streptococcus | Gram-positive | gramp1 |
| 053 | Streptococcus gordonii | Streptococcus | Gram-positive | gramp1 |
| 054 | Streptococcus hyointestinalis | Streptococcus | Gram-positive | gramp1 |
| 055 | Streptococcus intermedius | Streptococcus | Gram-positive | gramp1 |
| 056 | Streptococcus milleri | Streptococcus | Gram-positive | gramp1 |
| 057 | Streptococcus mitis | Streptococcus | Gram-positive | gramp1 |
| 058 | Streptococcus oralis | Streptococcus | Gram-positive | gramp1 |
| 059 | Streptococcus porcinus | Streptococcus | Gram-positive | gramp1 |
| 060 | Streptococcus pyogenes | Streptococcus | Gram-positive | gramp1 |
| 061 | Streptococcus salivarius | Streptococcus | Gram-positive | gramp1 |
| 062 | Streptococcus sanguinis | Streptococcus | Gram-positive | gramp1 |
| 063 | Streptococcus suis | Streptococcus | Gram-positive | gramp1 |
| 064 | Streptococcus uberis | Streptococcus | Gram-positive | gramp1 |
| 065 | Streptococcus equi subsp. | Streptococcus | Gram-positive | gramp1 |
| | Zooepidemicus | | | |
| 066 | Bacillus anthracis | Bacillus | Gram-positive | gramp1 |
| 067 | Bacillus cereus | Bacillus | Gram-positive | gramp1 |
| 068 | Bacillus coagulans | Bacillus | Gram-positive | gramp1 |
| 069 | Listeria monocytogenes | Listeria | Gram-positive | gramp1 |
| 070 | Clostridium perfringens | Clostridium | Gram-positive | gramp1 |
| 071 | Clostridium botulinum | Clostridium | Gram-positive | gramp1 |
| 072 | Neisseria gonorrhoeae | Neisseria | Gram-negative | gramn1 |
| 073 | Neisseria meningitidis | Neisseria | Gram-negative | gramn1 |
| 074 | Helicobacter pylori | Helicobacter | Gram-negative | gramn2 |
| 075 | Salmonella enteritidis | Salmonella | Gram-negative | gramn1 |
| 076 | Serratia marcescens | Serratia | Gram-negative | gramn1 |
| 077 | Staphylococcus schleiferi | Staphylococcus | Gram-positive | gramp1 |
| 078 | Eschenichia coil | Escherichia | Gram-negative | gramn1 |
| 079 | Empedobacter brevis | Empedobacter | Gram-negative | gramn1 |
| 080 | Stenotrophomonas maltophilia | Stenotrophomonas | Gram-negative | gramn1 |
| 081 | Staphylococcus saprophyticus | Staphylococcus | Gram-positive | gramp1 |
| 082 | Micrococcus luteus | Micrococcus | Gram-positive | gramp1 |
| 083 | Rothia sp. | Rothia | Gram-positive | gramp1 |
| 084 | Rothia mucilaginosa | Rothia | Gram-positive | gramp1 |
| 085 | Weissella confusa | Weisselia | Gram-positive | gramp1 |
| 086 | Lactobacillus casei | Lactobacillus | Gram-positive | gramp1 |
| 087 | Enterococcus casseliflavus | Enterococcus | Gram-positive | gramp1 |
| 088 | Aerococcus urinae | Aerococcus | Gram-positive | gramp1 |
| 089 | Aerococcus viridans | Aerococcus | Gram-positive | gramp1 |
| 090 | Listeria grayi | Listeria | Gram-positive | gramp1 |
| 091 | Corynebacterium diphtheriae | Corynebacterium | Gram-positive | gramp1 |
| 092 | Corynebactenium jeikeium | Corynebacterium | Gram-positive | gramp1 |
| 093 | Corynebacterium Group B | Corynebacterium | Gram-positive | gramp1 |
| 094 | Salmonella typhimurium | Salmonella | Gram-negative | gramn1 |
| 095 | Providencia alcalifaciens | Providencia | Gram-negative | gramn1 |
| 096 | Providencia rettgeri | Providencia | Gram-negative | gramn1 |
| 097 | Burkholderia cepacia | Burkholderia | Gram-negative | gramn1 |
| 098 | Burkholderia sp | Burkholderia | Gram-negative | gramn1 |
| 099 | Yersinia kristensenii | Yersinia | Gram-negative | gramn1 |
| 100 | Yersinia enterocolitica | Yersinia | Gram-negative | gramn1 |
| 101 | Citrobacter amalonaticus | Citrobacter | Gram-negative | gramn1 |
| 102 | Viridans group Streptococcus, | Viridans | Gram-negative | gramn1 |
| | not Milleri | | | |
| 103 | Proteus mirabilis | Proteus | Gram-negative | gramn1 |
| 104 | Proteus vulgaris | Proteus | Gram-negative | gramn1 |
| 105 | Enterobacter sakazakii | Enterobacter | Gram-negative | gramn1 |
| 106 | Brevundimonas diminuta | Brevundimonas | Gram-negative | gramn1 |
| 107 | Acinetobacter anitratus | Acinetobacter | Gram-negative | gramn1 |
| 108 | Haemophilus parainfluenzae | Haemophilus | Gram-negative | gramn1 |
| 109 | Haemophilus paraphrophilus | Haemophilus | Gram-negative | gramn1 |
| 110 | Neisseria meningitidis | Neisseria | Gram-negative | gramn1 |
| 111 | Neisseria gonorrhoeae | Neisseria | Gram-negative | gramn1 |
| 112 | Neisseria mucosa | Neisseria | Gram-negative | gramn1 |
| 113 | Neisseria lactamica | Neisseria | Gram-negative | gramn1 |
| 114 | Neisseria cinerea | Neisseria | Gram-negative | gramn1 |
| 115 | Bacteroides thetaiotaomicron | Bacteroides | Gram-negative | gramn1 |
| 116 | Bacteroides ovatus | Bacteroides | Gram-negative | gramn1 |
| 117 | Bacteroides fragilis | Bacteroides | Gram-negative | gramn1 |
| 118 | Afipia felis | Afipia | Gram-negative | gramn1 |
| 119 | Vibrio cholerae | Vibrio | Gram-negative | gramn1 |
| 120 | Eikenella corrodens | Eikenella | Gram-negative | gramn1 |
| 121 | Pasteurella multocida | Pasteurella | Gram-negative | gramn1 |
| 122 | Campylobacter jejuni | Campylobacter | Gram-negative | gramn2 |
| 123 | Serratia liquefaciens | Serratia | Gram-negative | gramn1 |
| 124 | Serratia odorifera | Serratia | Gram-negative | gramn1 |

When performing PCR using the primer set of the present invention, a target sequence region sought to be amplified is selected from nucleotide regions of a 16S rRNA gene of gram-negative bacteria corresponding to nucleotides 358-579 of SEQ ID NO 31, 823-1240 of SEQ ID NO 32, and 290-595 of SEQ ID NO 33, and nucleotide regions of a 16S rRNA gene of gram-positive bacteria corresponding to nucleotides 885-1169 of SEQ ID NO 34, 30-150 of SEQ ID NO 35, and 594-749 of SEQ ID NO 36.

The primer set of the present invention includes sequences for discriminating gram-negative bacteria and gram-positive bacteria. A primer set according to an exemplary embodiment of the present invention is presented in Table 2 below.

TABLE 2
Primer set according to an exemplary embodiment of the present invention

| Primer | SEQ ID NO | Remark |
|---|---|---|
| gramn1-F | 1 | Forward primer for amplification of nucleotide region corresponding |
| | | to nucleotides 358–579 of SEQ ID NO 31 |
| gramn1-R | 2 | Reverse primer for amplification of nucleotide region corresponding |
| | | to nucleotides 358–579 of SEQ ID NO 31 |
| gramn2-F | 3 | Forward primer for amplification of nucleotide region corresponding |
| | | to nucleotides 823–1240 of SEQ ID NO 32 |
| gramn2-R | 4 | Reverse primer for amplification of nucleotide region corresponding |
| | | to nucleotides 823–1240 of SEQ ID NO 32 |
| gramn3-F | 5 | Forward primer for amplification of nucleotide region corresponding |
| | | to nucleotides 290–595 of SEQ ID NO 33 |
| gramn3-R | 6 | Reverse primer for amplification of nucleotide region corresponding |
| | | to nucleotides 290–595 of SEQ ID NO 33 |
| gramp1-F | 7 | Forward primer for amplification of nucleotide region corresponding |
| | | to nucleotides 885–1169 of SEQ ID NO 34 |
| gramp1-R | 8 | Reverse primer for amplification of nucleotide region corresponding |
| | | to nucleotides 885–1169 of SEQ ID NO 34 |
| gramp2-F | 9 | Forward primer for amplification of nucleotide region corresponding |
| | | to nucleotides 30–150 of SEQ ID NO 35 |
| gramp2-R | 10 | Reverse primer for amplification of nucleotide region corresponding |
| | | to nucleotides 30–150 of SEQ ID NO 35 |
| gramp3-F | 11 | Forward primer for amplification of nucleotide region corresponding |
| | | to nucleotides 594–749 of SEQ ID NO 36 |
| gramp3-R | 12 | Reverse primer for amplification of nucleotide region corresponding |
| | | to nucleotides 594–749 of SEQ ID NO 36 |

As summarized in Table 2, the primer set of SEQ ID NOS: 1 and 2 is used for amplification of a nucleotide region of a 16S rRNA gene of gram-negative bacteria corresponding to nucleotides 358-579 of SEQ ID NO 31, the primer set of SEQ ID NOS: 3 and 4 is used for amplification of a nucleotide region of a 16S rRNA gene of gram-negative bacteria corresponding to nucleotides 823-1240 of SEQ ID NO 32, and the primer set of SEQ ID NOS: 5 and 6 is used for amplification of a nucleotide region of a 16S rRNA gene of gram-negative bacteria corresponding to nucleotides 290-595 of SEQ ID NO 33. Thus, products amplified using these primer sets are respectively 222 bp, 418 bp, and 306 bp in length, based on the consensus sequences. However, as a result of sequence variability of species within a given gram-negative bacterial group, the exact size of the product amplified by a given pair of primers will vary somewhat from the theoretical size predicted from the consensus sequence.

As summarized in Table 2, the primer set of SEQ ID NOS: 7 and 8 is used for amplification of a nucleotide region of a 16S rRNA gene of gram-positive bacteria corresponding to nucleotides 885-1169 of SEQ ID NO 34, the primer set of SEQ ID NOS: 9 and 10 is used for amplification of a nucleotide region of a 16S rRNA gene of gram-positive bacteria corresponding to nucleotides 30-150 of SEQ ID NO 35, and the primer set of SEQ ID NOS: 11 and 12 is used for amplification of a nucleotide region of a 16S rRNA gene of gram-positive bacteria corresponding to nucleotides 594-749 of SEQ ID NO 36. Thus, products amplified using these primer sets are respectively 285 bp, 121 bp, and 156 bp in length, based on the consensus sequences. As a result of sequence variability of species within a given gram-positive bacterial group, the exact size of the product amplified by a given pair of primers will vary somewhat from the theoretical size predicted from the consensus sequence.

In one embodiment, an oligonucleotide used as a probe or primer according to the invention can comprise a nucleotide analogue, e.g., a phosphorothioate, an alkylphosphorothioate, or a peptide nucleic acid. In some embodiments, the oligonucleotide used as a probe or primer comprises an intercalating agent.

The present invention also provides an oligonucleotide probe set capable of hybridizing with at least one target sequence selected from the group consisting of nucleotide regions of a 16S rRNA gene of gram-negative bacteria corresponding to nucleotides 358-579 of SEQ ID NO 31, 823-1240 of SEQ ID NO 32, and 290-595 of SEQ ID NO 33 and at least one target sequence selected from the group consisting of nucleotide regions of a 16S rRNA gene of gram-positive bacteria corresponding to nucleotides 885-1169 of SEQ ID NO 34, 30-150 of SEQ ID NO 35, and 594-749 of SEQ ID NO 36. The oligonucleotide probe set comprises: a first oligonucleotide probe selected from the group consisting of: an oligonucleotide probe capable of hybridizing with a nucleotide region of a 16S rRNA gene of gram-negative bacteria corresponding to positions 358-579 of SEQ ID NO 31, wherein the oligonucleotide probe consists of at least 10 contiguous nucleotides of a sequence selected from the group consisting of SEQ ID NOS: 13-15 and a complement of SEQ ID NOS: 13-15; an oligonucleotide probe capable of hybridizing with a nucleotide region of a 16S rRNA gene of gram-negative bacteria corresponding to positions 823-1240 of SEQ ID NO 32, wherein the oligonucleotide probe consists of at least 10 contiguous nucleotides of a sequence selected from the group consisting of SEQ ID NOS: 16-18 and a complement of SEQ ID NOS: 16-18; and an oligonucleotide probe capable of hybridizing with a nucleotide region of a 16S rRNA gene of gram-negative bacteria corresponding to positions 290-595 of SEQ ID NO 33, wherein the oligonucleotide probe consists of at least 10 contiguous nucleotides of a sequence selected from the group consisting of SEQ ID NOS: 19-21 and a complement of SEQ ID NOS: 19-21; and a second oligonucleotide probe selected from the group consisting of: an oligonucleotide probe capable of hybridizing with a nucleotide region of a 16S rRNA gene of gram-positive bacteria corresponding to positions 885-1169 of SEQ ID NO 34, wherein the oligonucleotide probe consists of at least 10 contiguous nucleotides of a sequence selected from the group consisting of SEQ ID NOS: 22-24 and a complement of SEQ ID NOS: 22-24; an oligonucleotide probe capable of hybridizing with a nucleotide region of a 16S rRNA gene of gram-positive bacteria corresponding to positions 30-150 of SEQ ID NO 35, wherein the oligonucleotide probe consists of at least 10 contiguous nucleotides of a sequence selected from the group consisting of SEQ ID NOS: 25-27 or a complement of SEQ ID NOS: 25-27; and an oligonucleotide probe capable of hybridizing with a nucleotide region of a 16S rRNA gene of gram-positive bacteria corresponding to positions 594-749 of SEQ ID NO 36, wherein the oligonucleotide probe consists of at least 10 contiguous nucleotides of a sequence selected from the group consisting of SEQ ID NOS: 28-30 or a complement of SEQ ID NOS: 28-30.

The probe set of the present invention can be an oligonucleotide probe set capable of hybridizing with at least one target sequence selected from the group consisting of nucleotide regions of a 16S rRNA gene of gram-negative bacteria corresponding to positions 358-579 of SEQ ID NO 31, 823-1240 of SEQ ID NO 32, and 290-595 of SEQ ID NO 33 and at least one target sequence selected from the group consisting of nucleotide regions of a 16S rRNA gene of gram-positive bacteria corresponding to positions 885-1169 of SEQ ID NO 34, 30-150 of SEQ ID NO 35, and 594-749 of SEQ ID NO 36, the oligonucleotide probe set including: the first oligonucleotide probe selected from the group consisting of: the oligonucleotide probe capable of hybridizing with a nucleotide region of a 16S rRNA gene of gram-negative bacteria corresponding to positions 358-579 of SEQ ID NO 31, wherein the oligonucleotide probe consists of a nucleotide sequence selected from the group consisting of the nucleotide sequences as set forth in SEQ ID NOS: 13-15; the oligonucleotide probe capable of hybridizing with a nucleotide region of a 16S rRNA gene of gram-negative bacteria corresponding to positions 823-1240 of SEQ ID NO 32, wherein the oligonucleotide probe consists of a nucleotide sequence selected from the group consisting of the nucleotide sequences as set forth in SEQ ID NOS: 16-18; and the oligonucleotide probe capable of hybridizing with a nucleotide region of a 16S rRNA gene of gram-negative bacteria corresponding to positions 290-595 of SEQ ID NO 33, wherein the oligonucleotide probe consists of a nucleotide sequence selected from the group consisting of the nucleotide sequences as set forth in SEQ ID NOS: 19-21; and the second oligonucleotide probe selected from the group consisting of: the oligonucleotide probe capable of hybridizing with a nucleotide region of a 16S rRNA gene of gram-positive bacteria corresponding to positions 885-1169 of SEQ ID NO 34, wherein the oligonucleotide probe consists of a nucleotide sequence selected from the group consisting of the nucleotide sequences as set forth in SEQ ID NOS: 22-24; the oligonucleotide probe capable of hybridizing with a nucleotide region of a 16S rRNA gene of gram-positive bacteria corresponding to positions 30-150 of SEQ ID NO 35, wherein the oligonucleotide probe consists of a nucleotide sequence selected from the group consisting of the nucleotide sequences as set forth in SEQ ID NOS: 25-27; and the oligonucleotide probe capable of hybridizing with a nucleotide region of a 16S rRNA gene of gram-positive bacteria corresponding to positions 594-749 of SEQ ID NO 36, wherein the oligonucleotide probe consists of a nucleotide sequence selected from the group consisting of the nucleotide sequences as set forth in SEQ ID NOS: 28-30.

The probe set of the present invention can be an oligonucleotide probe set capable of hybridizing with nucleotide regions of a 16S rRNA gene of gram-negative bacteria corresponding to nucleotides 358-579 of SEQ ID NO 31, 823-1240 of SEQ ID NO 32, and 290-595 of SEQ ID NO 33 and nucleotide regions of a 16S rRNA gene of gram-positive bacteria corresponding to nucleotides 885-1169 of SEQ ID NO 34, 30-150 of SEQ ID NO 35, and 594-749 of SEQ ID NO 36, the oligonucleotide probe set including: the oligonucleotide probe capable of hybridizing with a nucleotide region of a 16S rRNA gene of gram-negative bacteria corresponding to positions 358-579 of SEQ ID NO 31, wherein the oligonucleotide probe consists of a nucleotide sequence selected from the group consisting of the nucleotide sequences as set forth in SEQ ID NOS: 13-15; the oligonucleotide probe capable of hybridizing with a nucleotide region of a 16S rRNA gene of gram-negative bacteria corresponding to positions 823-1240 of SEQ ID NO 32, wherein the oligonucleotide probe consists of a nucleotide sequence selected from the group consisting of the nucleotide sequences as set forth in SEQ ID NOS: 16-18; the oligonucleotide probe capable of hybridizing with a nucleotide region of a 16S rRNA gene of gram-negative bacteria corresponding to positions 290-595 of SEQ ID NO 33, wherein the oligonucleotide probe consists of a nucleotide sequence selected from the group consisting of the nucleotide sequences as set forth in SEQ ID NOS: 19-21; the oligonucleotide probe capable of hybridizing with a nucleotide region of a 16S rRNA gene of gram-positive bacteria corresponding to positions 885-1169 of SEQ ID NO 34, wherein the oligonucleotide probe consists of a nucleotide sequence selected from the group consisting of the nucleotide sequences as set forth in SEQ ID NOS: 22-24; the oligonucleotide probe capable of hybridizing with a nucleotide region of a 16S rRNA gene of gram-positive bacteria corresponding to positions 30-150 of SEQ ID NO 35, wherein the oligonucleotide probe consists of a nucleotide sequence selected from the group consisting of the nucleotide sequences as set forth in SEQ ID NOS: 25-27; and the oligonucleotide probe capable of hybridizing with a nucleotide region of a 16S rRNA gene of gram-positive bacteria corresponding to positions 594-749 of SEQ ID NO 36, wherein the oligonucleotide probe consists of a nucleotide sequence selected from the group consisting of the nucleotide sequences as set forth in SEQ ID NOS: 28-30.

The probe set of the present invention specifically binds with at least one of the nucleotide regions of a 16S rRNA gene of gram-negative bacteria corresponding to nucleotides 358-579 of SEQ ID NO 31, 823-1240 of SEQ ID NO 32, and 290-595 of SEQ ID NO 33 and at least one of the nucleotide regions of a 16S rRNA gene of gram-positive bacteria corresponding to positions 885-1169 of SEQ ID NO 34, 30-150 of SEQ ID NO 35, and 594-749 of SEQ ID NO 36 which are amplified by PCR using a primer set according to an embodiment of the present invention. Therefore, the probe set of the present invention can discriminate gram-negative bacteria from gram-positive bacteria.

The probe set of the present invention includes sequences for discriminating gram-negative bacteria from gram-positive bacteria. A probe set according to an exemplary embodiment of the present invention is presented in Table 3 below.

TABLE 3
Probe set according to an exemplary embodiment of the present invention

| | | Gram positive/ | |
|---|---|---|---|
| SEQ ID NO | Probe | negative bacteria | Target nucleotide region of 16S rRNA gene |
| 13 | gramn1-pr1 | Negative | Nucleotides 358–579 of SEQ ID NO 31 |
| 14 | gramn1-pr2 | Negative | Nucleotides 358–579 of SEQ ID NO 31 |
| 15 | gramn1-pr3 | Negative | Nucleotides 358–579 of SEQ ID NO 31 |
| 16 | gramn2-pr1 | Negative | Nucleotides 823–1240 of SEQ ID NO 32 |
| 17 | gramn2-pr2 | Negative | Nucleotides 823–1240 of SEQ ID NO 32 |
| 18 | gramn2-pr3 | Negative | Nucleotides 823–1240 of SEQ ID NO 32 |
| 19 | gramn3-pr1 | Negative | Nucleotides 290–595 of SEQ ID NO 33 |
| 20 | gramn3-pr2 | Negative | Nucleotides 290–595 of SEQ ID NO 33 |
| 21 | gramn3-pr3 | Negative | Nucleotides 290–595 of SEQ ID NO 33 |
| 22 | gramp1-pr1 | Positive | Nucleotides 885–1169 of SEQ ID NO 34 |
| 23 | gramp1-pr2 | Positive | Nucleotides 885–1169 of SEQ ID NO 34 |
| 24 | gramp1-pr3 | Positive | Nucleotides 885–1169 of SEQ ID NO 34 |
| 25 | gramp2-pr1 | Positive | Nucleotides 30–150 of SEQ ID NO 35 |
| 26 | gramp2-pr2 | Positive | Nucleotides 30–150 of SEQ ID NO 35 |
| 27 | gramp2-pr3 | Positive | Nucleotides 30–150 of SEQ ID NO 35 |
| 28 | qramp3-pr1 | Positive | Nucleotides 594–749 of SEQ ID NO 36 |
| 29 | qramp3-pr2 | Positive | Nucleotides 594–749 of SEQ ID NO 36 |
| 30 | gramp3-pr3 | Positive | Nucleotides 594–749 of SEQ ID NO 36 |

As used herein, the term “probe” refers to a single-stranded nucleic acid sequence that can be hybridized with a complementary single-stranded target sequence to form a double-stranded molecule (hybrid).

The present invention also provides a microarray comprising a substrate on which at least one oligonucleotide probe set according to an embodiment of the present invention is immobilized.

As used herein, the term “microarray” refers to a high-density array of groups of polynucleotides immobilized on a substrate. Here, each group of polynucleotides on a microarray is immobilized on a predetermined region of the substrate. The microarray is well known in the art. Microarrays are disclosed in U.S. Pat. Nos. 5,445,934 and 5,744,305, the disclosures of which are incorporated herein in their entireties by reference. The oligonucleotide probe set used in the microarray is as described above.

As used herein, the term “substrate” refers to a substrate which can be coupled with an oligonucleotide probe under conditions that allow hybridization between the oligonucleotide probe and a target, for example, a DNA molecule in a sample, and achieve a low hybridization background. Conventionally, the substrate can be a microtiter plate, a membrane (e.g., nylon or nitrocellulose), a microsphere (bead), or a chip. A nucleic acid probe, before applied to or immobilized on a substrate, may be modified to facilitate probe immobilization or to enhance hybridization efficiency. The modification of the nucleic acid probe may include homopolymer tailing; coupling with a reactive functional group such as an aliphatic group, an NH₂ group, a SH group, or a carboxyl group; or coupling with biotin, a hapten, or a protein.

The present invention also provides methods of discriminating gram-negative bacteria from gram-positive bacteria in a sample.

In an embodiment, the method comprises contacting a sample with the oligonucleotide probe set disclosed herein to allow hybridization between a target sequence of the sample and the first and second oligonucleotide probes; and detecting hybridization between the first and second oligonucleotide probes and the target sequence of the sample to discriminate gram-negative bacteria from gram-positive bacteria in the sample.

In an embodiment, the method comprises using genomic DNA in a sample as a template and a first oligonucleotide primer set specific for amplifying a target region specific to a gram-negative bacterium in a 16S rRNA gene and a second oligonucleotide primer set specific for amplifying a target region specific to a gram-positive bacterium in a 16S rRNA gene in an amplification reaction; and detecting the gram-negative bacterium in the sample when the gram-negative bacterium-specific target sequence is amplified and detecting the gram-positive bacterium in the sample when the gram-positive bacterium-specific target sequence is amplified.

As used herein, the term “hybridization” refers to the binding of two complementary strands of nucleic acid to form a double-stranded molecule (hybrid).

In the method of the present invention, the hybridization may be performed under a high stringency hybridization condition. For example, the high stringency hybridization condition may be in a 0.12M phosphate buffer including equal moles of Na₂HPO₄ and NaH₂PO₄, 1 mM EDTA, and 0.02% sodium dodecylsulfate at 65° C.

As used herein, “stringency” is the term used to describe the temperature and solvent composition during hybridization and the subsequent processes. Under high stringency conditions, highly homologous nucleic acid hybrids will be formed and hybrids with an insufficient degree of complementary will not be formed. Accordingly, the stringency of the assay conditions determines the amount of hybridization between two nucleic acid strands. Stringency is chosen to maximize the difference in stability between probe-target hybrids and probe-non-target hybrids.

In the method of the present invention, the sample can include amplification products obtained by amplification of template using a primer set according to an embodiment of the present invention as primers and DNAs derived from gram-negative bacteria and/or gram-positive bacteria as templates.

A nucleic acid used in the method of the present invention can be selected from the group consisting of chromosomal DNA, cDNA, or a fragment thereof.

As used herein, the “PCR” refers to a polymerase chain reaction. It is a method for amplifying a target nucleic acid from a primer pair specifically bound to the target nucleic acid using a DNA polymerase. PCR is well known in the art and can be performed using a commercially available kit. PCR includes single PCR, for amplifying only a single target, and multiplex PCR, for simultaneously amplifying multiple targets. A plurality of primer pairs is used in a multiplex PCR.

In the present invention, multiplex PCR can be performed using at least one primer set specific to gram-negative bacteria and at least one primer set specific to gram-positive bacteria. Preferably, a multiplex PCR may be performed using three primer sets specific to gram-negative bacteria and three primer sets specific to gram-positive bacteria. In this case, more bacterial species can be discriminated.

The amplification of a target nucleic acid can also be performed using any appropriate method known in the art, e.g., ligase chain reaction, nucleic acid sequence-based amplification, transcription-based amplification system, strand displacement amplification, Qβ replicase amplification, or other nucleic acid amplification methods, in addition to PCR.

In the method of the present invention, the target sequence may be labeled with a detectable labeling material. For example, the labeling material may be a fluorescent material, a phosphorescent material, or a radioactive material, but the present invention is not limited thereto. Preferably, the labeling material may be Cy-5 or Cy-3. When the target sequence is amplified, for example, by PCR, using primers which are labeled with Cy-5 or Cy-3 at the 5′ ends, the target sequence can be labeled with the detectable labeling material. When performing amplification using a radioactive material, a radioisotope such as ³²P or ³⁵S is incorporated into one or more of the NTPs or dNTPs added to the amplification reaction such that the resulting amplified product is then labeled with the radioisotope.

In the method of the present invention, the oligonucleotide probe set can be immobilized on a substrate of a microarray. The oligonucleotide probe set immobilized on the substrate of the microarray is as described above.

In the method of the present invention, gram-negative bacteria can be distinguished from gram-positive bacteria by labeling an amplification product, for example a PCR product, with a detectable signal-emitting material, hybridizing the labeled amplification product with the oligonucleotide probe set, and detecting a signal generated from the hybridization product. The detectable signal can be an optical signal or an electrical signal, but the present invention is not limited thereto. An optically active material can be a fluorescent material or a phosphorescent material. The fluorescent material can be fluorescein, Cy-5, or Cy-3. The amplification product can be unlabeled, or labeled with the detectable signal-emitting material before or after hybridization. In the case where the amplification product is unlabeled, hybridization between the amplification product and the oligonucleotide probe set can be detected by an electrical signal, but the present invention is not limited thereto.

The present invention also provides a kit for discriminating gram-negative bacteria and gram-positive bacteria, the kit comprises a primer set according to an embodiment of the present invention. The kit of the present invention can includereagents for performing amplification. In an embodiment, the reagents can include DNA polymerase, dNTPs, a buffer, and the like. The kit of the present invention can further include an instruction manual specifying optimal reaction conditions.

The kit of the present invention may further include an oligonucleotide probe set according to an embodiment of the present invention. The kit of the present invention can discriminate gram-negative bacteria and gram-positive bacteria using a primer set according to an embodiment of the present invention. However, in order to achieve more accurate discrimination, a probe set capable of specifically, hybridizing with a product amplified using the primer set may also be used in the kit of the present invention.

Hereinafter, the present invention will be described more specifically with reference to the following examples. The following examples are for illustrative purposes only and are not intended to limit the scope of the invention.

### EXAMPLES

### Example 1

### Selection of Primers for Discriminating Gram-Negative Bacteria and Gram-Positive Bacteria

In Example 1, primers for discriminating gram-negative bacteria and gram-positive bacteria were selected. For this, target sequences specifically found in the 16S rRNA genes of gram-negative bacteria and gram-positive bacteria were selected and primer sets capable of amplifying the target sequences were designed.

First, gram-negative and gram-positive bacterial sequences were acquired from Genbank, and 16S rRNA gene sequences specifically found in gram-negative bacteria or in gram-positive bacteria were selected using the program DNASTAR. The bacterial species used are as described above in Table 1. The selected regions of the 16S rRNA gene with sequences specific to the gram-negative bacteria were the nucleotide regions corresponding to nucleotides 358-579 of SEQ ID NO 31, 823-1240 of SEQ ID NO 32, and 290-595 of SEQ ID NO 33. The selected regions of the 16S rRNA gene specific to the gram-positive bacteria were the nucleotide regions corresponding to nucleotides 885-1169 of SEQ ID NO 34, 30-150 of SEQ ID NO 35, and 594-749 of SEQ ID NO 36.

Subsequently, six oligonucleotide primer sets were designed using the sequence regions specific to gram-negative or gram-positive bacteria: an oligonucleotide primer set having sequences as set forth in SEQ ID NOS: 1 and 2, an oligonucleotide primer set having sequences as set forth in SEQ ID NOS: 3 and 4, an oligonucleotide primer set having sequences as set forth in SEQ ID NOS: 5 and 6, an oligonucleotide primer set having sequences as set forth in SEQ ID NOS: 7 and 8, an oligonucleotide primer set having sequences as set forth in SEQ ID NOS: 9 and 10, and an oligonucleotide primer set having sequences as set forth in SEQ ID NOS: 11 and 12. Each of these oligonucleotide primer sets can amplify one of the selected target regions of the 16S rRNA gene in gram-negative and gram-positive bacterial species, as summarized in Table 2.

### Example 2

### Amplification of the 16S rRNA Gene of Gram-Negative and Gram-Positive Bacterial Species Using the Primer Sets of the Present Invention

The 16S rRNA genes of gram-negative and gram-positive bacterial species were amplified using the six primer sets designed in Example 1.

First, single PCR was performed to determine if each target sequence was specifically amplified. Single PCR used, as templates, genomic DNAs of three gram-negative bacterial species, one representing each of the three gram-negative detection groups, and three gram-positive bacteria species, one representing each of the three gram-positive detection groups, presented in Table 4 below, and, as primers, the six primer sets designed in Example 1.

TABLE 4
Bacterial species used in PCR

| # | Bacterial species | Primer set |
|---|---|---|
| 013 | klebsiella pneumoniae | gramn1 |
| 012 | Helicobacter pylori | gramn2 |
| 004 | Chlamydophila pneumoniae | gramn3 |
| 026 | Staphylococcus aureus | gramp1 |
| 027 | Staphylococcus cohnii | gramp2 |
| 017 | Mycoplasma pneumophila | gramp3 |

The single PCR was performed in 20 μl of a PCR reaction mixture containing 2 μl of genomic DNA (extracted using a G-spin genomic DNA extraction kit, iNtRON). Final concentrations of various components in the PCR reaction mixture were: 1.5 mM of MgCl₂, 250 mM of each dNTP, 10 mM tris-HCl (pH 9.0), 1 unit of Taq polymerase, and about 2 pmol of each primer. The amplification program was the following: 25 cycles of denaturation at 95° C. for 10 seconds, annealing at 60° C. for 10 seconds, and extension at 60° C. for 13 seconds.

Next, multiplex PCR was performed using, as templates, genomic DNAs of the three gram-negative bacterial species and the three gram-positive bacterial species, and, as primers, the six primer sets designed in Example 1. The PCR mix for the multiplex PCR was made up to a total volume of 50 μl, containing 10.5 μl of distilled water, 7.5 μl of 10× buffer (750 mM Tris-HCl, pH 9; 150 mM (NH₄)₂SO₄, 25 mM MgCl₂, 1 mg/ml BSA), 1 μl of 200 μM dNTP (each), 20 μl of 400 nM end-labeled primer (each, Bioneer, Korea), 5 μl of extracted genomic DNA, and 1 μl of Taq polymerase (5 units).

The multiplex PCR was performed as follows: initial denaturation at 95° C. for one minute; 25 cycles of denaturation at 95° C. for 5 seconds, annealing at 62° C. for 13 seconds, and extension at 72° C. for 15 seconds; and extension at 72° C. for one minute.

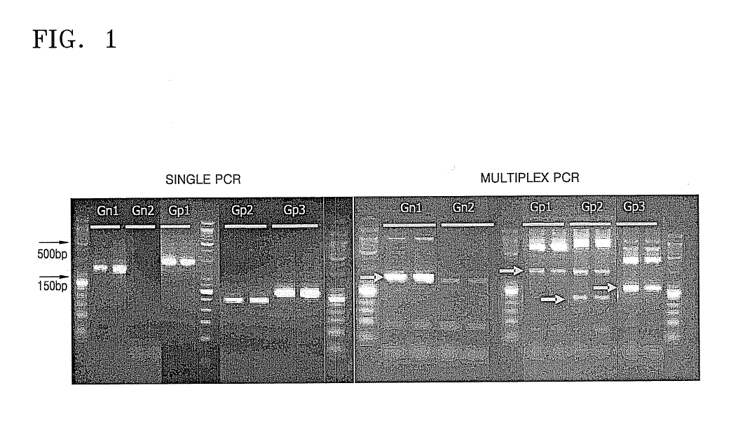

The PCR products of both the single and multiplex PCR were analyzed by electrophoresis on an agarose gel, and detected by staining with ethidium bromide. FIG. 1 shows a photograph of the agarose gel electrophoretic analysis of the PCR products obtained by the single PCR and the multiplex PCR. In FIG. 1, “Gn1” and “Gn2” are lanes for PCR results obtained by PCR using genomic DNAs of Klebsiella pneumoniae and Helicobacter pylori as templates and the gramn1 and gramn2 primer sets as primers, respectively. “Gp1”, “Gp2”, and “Gp3” are lanes for PCR results obtained by PCR using genomic DNAs of Staphylococcus aureus, Staphylococcus cohnii, and Mycoplasma pneumophila as templates and the gramp1, gramp2, and gramp3 primer sets as primers, respectively. Referring to FIG. 1, with respect to the single PCR results, in lanes other than lane Gn2, desired PCR products were detected. No PCR product was detected in lane Gn2. Helicobacter pylori is a strain which shows severe sequence variations; no target was amplified under PCR conditions of the present invention. Thus, it is likely that PCR conditions needed to be optimized to amplify the target sequence in Helicobacter pylori.

The results of the multiplex PCR, which was performed by simultaneously adding the six primer sets to each bacterial species, generated similar results as those of the single PCR. This shows that primer sets are specific to target bacterial species. Thus, it can be seen that the use of six primer sets according to an embodiment of the present invention enables one to clearly discriminate gram-negative bacteria and gram-positive bacteria. That is, when performing multiplex PCR using a template DNA derived from unknown bacterial species and six primer sets according to an embodiment of the present invention and measuring the size of a PCR product using agarose gel electrophoresis, it is possible to determine if the unknown bacterial species is gram-negative or gram-positive.

### Example 3

### Design of Probes According to the Present Invention

In Example 3, probes were designed to detect the products of amplification resulting from using the primer sets disclosed in Table 2. The probes were selected to be specific for the species group-specific amplified regions of the 16S rRNA genes of gram-negative or gram-positive bacterial species using the DNASTAR program. The selected probes are summarized in Table 3 above.

The probes are immobilized on a microarray.

The PCR products obtained in Example 2 are applied to the microarray to allow hybridization between the PCR products and the probes immobilized on the microarray. Hybridization signals are detected by conventional methods to determine the presence of specific PCR products.

According to a primer set of the present invention, gram-negative bacteria and gram-positive bacteria can be efficiently discriminated.

According to a method of the present invention, gram-negative bacteria and gram-positive bacteria can be discriminated with high specificity.

The terminology used herein is for the purpose of describing particular embodiments only and is not intended to be limiting of the invention. The terms “a” and “an” do not denote a limitation of quantity, but rather denote the presence of at least one of the referenced item. The term “or” means “and/or”. The terms “comprising”, “having”, “including”, and “containing” are to be construed as open-ended terms (i.e., meaning “including, but not limited to”).

Recitation of ranges of values are merely intended to serve as a shorthand method of referring individually to each separate value falling within the range, unless otherwise indicated herein, and each separate value is incorporated into the specification as if it were individually recited herein. The endpoints of all ranges are included within the range and independently combinable.

All methods described herein can be performed in a suitable order unless otherwise indicated herein or otherwise clearly contradicted by context. The use of any and all examples, or exemplary language (e.g., “such as”), is intended merely to better illustrate the invention and does not pose a limitation on the scope of the invention unless otherwise claimed. No language in the specification should be construed as indicating any non-claimed element as essential to the practice of the invention as used herein. Unless defined otherwise, technical and scientific terms used herein have the same meaning as is commonly understood by one of skill in the art to which this invention belongs.

Preferred embodiments of this invention are described herein, including the best mode known to the inventors for carrying out the invention. Variations of those preferred embodiments may become apparent to those of ordinary skill in the art upon reading the foregoing description. The inventors expect skilled artisans to employ such variations as appropriate, and the inventors intend for the invention to be practiced otherwise than as specifically described herein. Accordingly, this invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context. While the present invention has been particularly shown and described with reference to exemplary embodiments thereof, it will be understood by those of ordinary skill in the art that various changes in form and details may be made therein without departing from the spirit and scope of the present invention as defined by the following claims.

What is claimed is:

1. An oligonucleotide primer set comprising:
a first oligonucleotide set comprising
an oligonucleotide set comprising an oligonucleotide consisting of at least 10 contiguous nucleotides of SEQ ID NO: 1 and an oligonucleotide consisting of at least 10 contiguous nucleotides of SEQ ID NO: 2;
an oligonucleotide set comprising an oligonucleotide consisting of at least 10 contiguous nucleotides of SEQ ID NO: 3 and at least one oligonucleotide consisting of at least 10 contiguous nucleotides of SEQ ID NO: 4; or
an oligonucleotide set comprising at least one oligonucleotide consisting of at least 10 contiguous nucleotides of SEQ ID NO: 5 and at least one oligonucleotide consisting of at least 10 contiguous nucleotides of SEQ ID NO: 6, and
a second oligonucleotide set comprising
an oligonucleotide set comprising at least one oligonucleotide consisting of at least 10 contiguous nucleotides of SEQ ID NO: 7 and at least one oligonucleotide consisting of at least 10 contiguous nucleotides of SEQ ID NO: 8;
an oligonucleotide set comprising at least one oligonucleotide consisting of at least 10 contiguous nucleotides of SEQ ID NO: 9 and at least one oligonucleotide consisting of at least 10 contiguous nucleotides of SEQ ID NO: 10; or
an oligonucleotide set comprising at least one oligonucleotide consisting of of at least 10 contiguous nucleotides of SEQ ID NO: 11 and at least one oligonucleotide consisting of at least 10 contiguous nucleotides of SEQ ID NO: 12,
wherein the oligonucleotide primer set amplifies a target sequence of a 16S rRNA gene in gram-negative bacteria and a target sequence of a 16S rRNA gene in gram-positive bacteria.

2. The oligonucleotide primer set of claim 1, wherein the target sequence in gram-negative bacteria is a nucleotide region of a 16S rRNA gene of gram-negative bacteria corresponding to nucleotides 358-579 of SEQ ID NO 31, 823-1240 of SEQ ID NO 32, or 290-595 of SEQ ID NO 33 and the target sequence in gram-positive bacteria is a nucleotide region of a 16S rRNA gene of gram-positive bacteria corresponding to nucleotides 885-1169 of SEQ ID NO 34, 30-150 of SEQ ID NO 35, or 594-749 of SEQ ID NO 36.

3. The oligonucleotide primer set of claim 1, wherein the first oligonucleotide set comprises an oligonucleotide consisting of at least 10 contiguous nucleotides of SEQ ID NO: 1 and an oligonucleotide consisting of at least 10 contiguous nucleotides of SEQ ID NO: 2, and
wherein the first oligonucleotide set amplifies a region of the 16S rRNA gene of gram-negative bacteria corresponding to nucleotides 358-579 of SEQ ID NO 31.

4. The oligonucleotide primer set of claim 1, wherein the first oligonucleotide set comprises an oligonucleotide consisting of at least 10 contiguous nucleotides of SEQ ID NO: 3 and an oligonucleotide consisting of at least 10 contiguous nucleotides of SEQ ID NO: 4, and
wherein the first oligonucleotide set amplifies a region of the 16S rRNA gene of gram-negative bacteria corresponding to nucleotides 823-1240 of SEQ ID NO 32.

5. The oligonucleotide primer set of claim 1, wherein the first oligonucleotide set comprises an oligonucleotide consisting of at least 10 contiguous nucleotides of SEQ ID NO: 5 and an oligonucleotide consisting of at least 10 contiguous nucleotides of SEQ ID NO: 6, and
wherein the first oligonucleotide set amplifies a region of the 16S rRNA gene of gram-negative bacteria corresponding to nucleotides 290-595 of SEQ ID NO 33.

6. The oligonucleotide primer set of claim 1, wherein the secondoligonucleotide set comprises an oligonucleotide consisting of at least 10 contiguous nucleotides of SEQ ID NO: 7 and an oligonucleotide consisting of at least 10 contiguous nucleotides of SEQ ID NO: 8, and
wherein the second oligonucleotide set amplifies a region of the 16S rRNA gene of gram-positive bacteria corresponding to nucleotides 885-1169 of SEQ ID NO 34.

7. The oligonucleotide primer set of claim 1, wherein the second oligonucleotide set comprises an oligonucleotide consisting of at least 10 contiguous nucleotides of SEQ ID NO: 9 and an oligonucleotide consisting of at least 10 contiguous nucleotides of SEQ ID NO: 10, and
wherein the second oligonucleotide set amplifies a region of the 16S rRNA gene of gram-positive bacteria corresponding to nucleotides 30-150 of SEQ ID NO 35.

8. The oligonucleotide primer set of claim 1, wherein the second oligonucleotide set comprises an oligonucleotide consisting of at least 10 contiguous nucleotides of SEQ ID NO: 11 and an oligonucleotide consisting of at least 10 contiguous nucleotides of SEQ ID NO: 12, and
wherein the second oligonucleotide set amplifies a region of the 16S rRNA gene of gram-positive bacteria corresponding to nucleotides 594-749 of SEQ ID NO 36.

9. The oligonucleotide primer set of claim 1, comprising:
a first oligonucleotide set selected from the group consisting of
the oligonucleotide set comprising an oligonucleotide consisting of the nucleotide sequence as set forth in SEQ ID NO: 1 and an oligonucleotide consisting of the nucleotide sequence as set forth in SEQ ID NO: 2;
the oligonucleotide set comprising an oligonucleotide consisting of the nucleotide sequence as set forth in SEQ ID NO: 3 and an oligonucleotide consisting of the nucleotide sequence as set forth in SEQ ID NO: 4; and
the oligonucleotide set comprising an oligonucleotide consisting of the nucleotide sequence as set forth in SEQ ID NO: 5 and an oligonucleotide consisting of the nucleotide sequence as set forth in SEQ ID NO: 6, and
a second oligonucleotide set selected from the group consisting of
the oligonucleotide set comprising an oligonucleotide consisting of the nucleotide sequence as set forth in SEQ ID NO: 7 and an oligonucleotide consisting of the nucleotide sequence as set forth in SEQ ID NO: 8;
the oligonucleotide set comprising an oligonucleotide consisting of the nucleotide sequence as set forth in SEQ ID NO: 9 and an oligonucleotide consisting of the nucleotide sequence as set forth in SEQ ID NO: 10; and
the oligonucleotide set comprising an oligonucleotide consisting of the nucleotide sequence as set forth in SEQ ID NO: 11 and an oligonucleotide consisting of the nucleotide sequence as set forth in SEQ ID NO: 12,
wherein the oligonucleotide primer set amplifies a first target sequence selected from the group consisting of nucleotide regions of the 16S rRNA gene of gram-negative bacteria corresponding to positions 358-579 of SEQ ID NO 31, 823-1240 of SEQ ID NO 32, and 290-595 of SEQ ID NO 33 and a second target sequence selected from the group consisting of nucleotide regions of the 16S rRNA gene of gram-positive bacteria corresponding to positions 885-1169 of SEQ ID NO 34, 30-150 of SEQ ID NO 35, and 594-749 of SEQ ID NO 36.

10. The oligonucleotide primer set of claim 9, which comprises
the oligonucleotide set comprising an oligonucleotide consisting of SEQ ID NO: 1 and an oligonucleotide consisting of SEQ ID NO: 2;
the oligonucleotide set comprising an oligonucleotide consisting of SEQ ID NO: 3 and an oligonucleotide consisting of SEQ ID NO: 4;
the oligonucleotide set comprising an oligonucleotide consisting of SEQ ID NO: 5 and an oligonucleotide consisting of SEQ ID NO: 6;
the oligonucleotide set comprising an oligonucleotide consisting of SEQ ID NO: 7 and an oligonucleotide consisting of SEQ ID NO: 8;
the oligonucleotide set comprising an oligonucleotide consisting of SEQ ID NO: 9 and an oligonucleotide consisting of SEQ ID NO: 10; and
the oligonucleotide set comprising an oligonucleotide consisting of SEQ ID NO: 11 and an oligonucleotide consisting of SEQ ID NO: 12,
wherein the oligonucleotide primer set amplifies nucleotide regions of the 16S rRNA gene of gram-negative bacteria corresponding to positions 358-579 of SEQ ID NO 31, 823-1240 of SEQ ID NO 32, and 290-595 of SEQ ID NO 33 and nucleotide regions of the 16S rRNA gene of gram-positive bacteria corresponding to positions 885-1169 of SEQ ID NO 34, 30-150 of SEQ ID NO 35, and 594-749 of SEQ ID NO 36.

11. An oligonucleotide probe set comprising:
a first oligonucleotide probe selected from the group consisting of:
an oligonucleotide probe capable of hybridizing with a nucleotide region of a 16S rRNA gene of gram-negative bacteria corresponding to positions 358-579 of SEQ ID NO 31, wherein the oligonucleotide probe consists of at least 10 contiguous nucleotides of a sequence selected from the group consisting of SEQ ID NOS: 13-15 and a complement of SEQ ID NOS: 13-15;
an oligonucleotide probe capable of hybridizing with a nucleotide region 32 of a 16S rRNA gene of gram-negative bacteria corresponding to positions 823-1240 of SEQ ID NO 32, wherein the oligonucleotide probe consists of at least 10 contiguous nucleotides of a sequence selected from the group consisting of SEQ ID NOS: 16-18 and a complement of SEQ ID NOS: 16-18; and
an oligonucleotide probe capable of hybridizing with a nucleotide region of a 16S rRNA gene of gram-negative bacteria corresponding to positions 290-595 of SEQ ID NO 33, wherein the oligonucleotide probe consists of at least 10 contiguous nucleotides of a sequence selected from the group consisting of SEQ ID NOS: 19-21 and a complement of SEQ ID NOS: 19-21, and
a second oligonucleotide probe selected from the group consisting of:
an oligonucleotide probe capable of hybridizing with a nucleotide region 34 of a 16S rRNA gene of gram-positive bacteria corresponding to positions 885-1169 of SEQ ID NO 34, wherein the oligonucleotide probe consists of at least 10 contiguous nucleotides of a sequence selected from the group consisting of SEQ ID NOS: 22-24 and a complement of SEQ ID NOS: 22-24;
an oligonucleotide probe capable of hybridizing with a nucleotide region of a 16S rRNA gene of gram-positive bacteria corresponding to positions 30-150 of SEQ ID NO 35, wherein the oligonucleotide probe consists of at least 10 contiguous nucleotides of a sequence selected from the group consisting of SEQ ID NOS: 25-27 or a complement of SEQ ID NOS: 25-27; and
an oligonucleotide probe capable of hybridizing with a nucleotide region of a 16S rRNA gene of gram-positive bacteria corresponding to positions 594-749 of SEQ ID NO 36, wherein the oligonucleotide probe consists of at least 10 contiguous nucleotides of a sequence selected from the group consisting of SEQ ID NOS: 28-30 or a complement of SEQ ID NOS: 28-30.

12. The oligonucleotide probe set of claim 11, comprising:
the first oligonucleotide probe selected from the group consisting of:
the oligonucleotide probe capable of hybridizing with a nucleotide region of a 16S rRNA gene of gram-negative bacteria corresponding to positions 358-579 of SEQ ID NO 31, wherein the oligonucleotide probe consists of a nucleotide sequence selected from the group consisting of the nucleotide sequences as set forth in SEQ ID NOS: 13-15;
the oligonucleotide probe capable of hybridizing with a nucleotide region of a 16S rRNA gene of gram-negative bacteria corresponding to positions 823-1240 of SEQ ID NO 32, wherein the oligonucleotide probe consists of a nucleotide sequence selected from the group consisting of the nucleotide sequences as set forth in SEQ ID NOS: 16-18; and
the oligonucleotide probe capable of hybridizing with a nucleotide region of a 16S rRNA gene of gram-negative bacteria corresponding to positions 290-595 of SEQ ID NO 33, wherein the oligonucleotide probe consists of a nucleotide sequence selected from the group consisting of the nucleotide sequences as set forth in SEQ ID NOS: 19-21, and
the second oligonucleotide probe selected from the group consisting of:
the oligonucleotide probe capable of hybridizing with a nucleotide region of a 16S rRNA gene of gram-positive bacteria corresponding to positions 885-1169 of SEQ ID NO 34, wherein the oligonucleotide probe consists of a nucleotide sequence selected from the group consisting of the nucleotide sequences as set forth in SEQ ID NOS: 22-24;
the oligonucleotide probe capable of hybridizing with a nucleotide region of a 16S rRNA gene of gram-positive bacteria corresponding to positions 30-150 of SEQ ID NO 35, wherein the oligonucleotide probe consists of a nucleotide sequence selected from the group consisting of the nucleotide sequences as set forth in SEQ ID NOS: 25-27; and
the oligonucleotide probe capable of hybridizing with a nucleotide region of a 16S rRNA gene of gram-positive bacteria corresponding to positions 594-749 of SEQ ID NO 36, wherein the oligonucleotide probe consists of a nucleotide sequence selected from the group consisting of the nucleotide sequences as set forth in SEQ ID NOS: 28-30.

13. The oligonucleotide probe set of claim 12, comprising:
the oligonucleotide probe capable of hybridizing with a nucleotide region of a 16S rRNA gene of gram-negative bacteria corresponding to positions 358-579 of SEQ ID NO 31, wherein the oligonucleotide probe consists of a nucleotide sequence selected from the group consisting of the nucleotide sequences as set forth in SEQ ID NOS: 13-15;
the oligonucleotide probe capable of hybridizing with a nucleotide region of a 16S rRNA gene of gram-negative bacteria corresponding to positions 823-1240 of SEQ ID NO 32, wherein the oligonucleotide probe consists of a nucleotide sequence selected from the group consisting of the nucleotide sequences as set forth in SEQ ID NOS: 16-18;
the oligonucleotide probe capable of hybridizing with a nucleotide region of a 16S rRNA gene of gram-negative bacteria corresponding to positions 290-595 of SEQ ID NO 33, wherein the oligonucleotide probe consists of a nucleotide sequence selected from the group consisting of the nucleotide sequences as set forth in SEQ ID NOS: 19-21;
the oligonucleotide probe capable of hybridizing with a nucleotide region of a 16S rRNA gene of gram-positive bacteria corresponding to positions 885-1169 of SEQ ID NO 34, wherein the oligonucleotide probe consists of a nucleotide sequence selected from the group consisting of the nucleotide sequences as set forth in SEQ ID NOS: 22-24;
the oligonucleotide probe capable of hybridizing with a nucleotide region of a 16S rRNA gene of gram-positive bacteria corresponding to positions 30-150 of SEQ ID NO 35, wherein the oligonucleotide probe consists of a nucleotide sequence selected from the group consisting of the nucleotide sequences as set forth in SEQ ID NOS: 25-27; and
the oligonucleotide probe capable of hybridizing with a nucleotide region of a 16S rRNA gene of gram-positive bacteria corresponding to positions 594-749 of SEQ ID NO 36, wherein the oligonucleotide probe consists of a nucleotide sequence selected from the group consisting of the nucleotide sequences as set forth in SEQ ID NOS: 28-30.

14. A microarray, comprising
a substrate on which the oligonucleotide probe set of claim 11 is immobilized.

15. A method of discriminating gram-negative bacteria from gram-positive bacteria in a sample, the method comprising:
contacting a sample with the oligonucleotide probe set of claim 11 to allow hybridization between a target sequence of the sample and the first and second oligonucleotide probes; and
detecting hybridization between the first and second oligonucleotide probes and the target sequence of the sample to discriminate gram-negative bacteria from gram-positive bacteria in the sample.

16. The method of claim 15, wherein the sample comprises PCR products obtained by PCR using as primers an oligonucleotide primer set comprising:
a first oligonucleotide set comprising
an oligonucleotide set comprising an oligonucleotide consisting of at least 10 contiguous nucleotides of SEQ ID NO: 1 and an oligonucleotide consisting of at least 10 contiguous nucleotides of SEQ ID NO: 2;
an oligonucleotide set comprising an oligonucleotide consisting of at least 10 contiguous nucleotides of SEQ ID NO: 3 and at least one oligonucleotide consisting of at least 10 contiguous nucleotides of SEQ ID NO: 4; or
an oligonucleotide set comprising at least one oligonucleotide consisting of at least 10 contiguous nucleotides of SEQ ID NO: 5 and at least one oligonucleotide consisting of at least 10 contiguous nucleotides of SEQ ID NO: 6, and
a second oligonucleotide set comprising
an oligonucleotide set comprising at least one oligonucleotide consisting of at least 10 contiguous nucleotides of SEQ ID NO: 7 and at least one oligonucleotide consisting of at least 10 contiguous nucleotides of SEQ ID NO: 8;
an oligonucleotide set comprising at least one oligonucleotide consisting of at least 10 contiguous nucleotides of SEQ ID NO: 9 and at least one oligonucleotide consisting of at least 10 contiguous nucleotides of SEQ ID NO: 10; or
an oligonucleotide set comprising at least one oligonucleotide consisting of of at least 10 contiguous nucleotides of SEQ ID NO: 11 and at least one oligonucleotide consisting of at least 10 contiguous nucleotides of SEQ ID NO: 12,
wherein the oligonucleotide primer set amplifies a target sequence of a 16S rRNA gene in gram-negative bacteria and a target sequence of a 16S rRNA gene in gram-positive bacteria; and
DNAs derived from gram-negative bacteria and gram-positive bacteria as templates.

17. The method of claim 15, wherein the target sequence is labeled with a detectable labeling material.

18. The method of claim 17, wherein the labeling material is a fluorescent material, a phosphorescent material, or a radioactive material.

19. The method of claim 15, wherein the oligonucleotide probe set is immobilized on a microarray substrate.

20. A kit for discriminating gram-negative bacteria and gram-positive bacterial, comprising the primer set of claim 1.

21. The kit of claim 20, further comprising an oligonucleotide probe set comprising:
a first oligonucleotide probe selected from the group consisting of:
an oligonucleotide probe capable of hybridizing with a nucleotide region of a 16S rRNA gene of gram-negative bacteria corresponding to positions 358-579 of SEQ ID NO 31, wherein the oligonucleotide probe consists of at least 10 contiguous nucleotides of a sequence selected from the group consisting of SEQ ID NOS: 13-15 and a complement of SEQ ID NOS: 13-15;
an oligonucleotide probe capable of hybridizing with a nucleotide region of a 16S rRNA gene of gram-negative bacteria corresponding to positions 823-1240 of SEQ ID NO 32, wherein the oligonucleotide probe consists of at least 10 contiguous nucleotides of a sequence selected from the group consisting of SEQ ID NOS: 16-18 and a complement of SEQ ID NOS: 16-18; and
an oligonucleotide probe capable of hybridizing with a nucleotide region of a 16S rRNA gene of gram-negative bacteria corresponding to positions 290-595 of SEQ ID NO 33, wherein the oligonucleotide probe consists of at least 10 contiguous nucleotides of a sequence selected from the group consisting of SEQ ID NOS: 19-21 and a complement of SEQ ID NOS: 19-21, and
a second oligonucleotide probe selected from the group consisting of:
an oligonucleotide probe capable of hybridizing with a nucleotide region of a 16S rRNA gene of gram-positive bacteria corresponding to positions 885-1169 of SEQ ID NO 34, wherein the oligonucleotide probe consists of at least 10 contiguous nucleotides of a sequence selected from the group consisting of SEQ ID NOS: 22-24 and a complement of SEQ ID NOS: 22-24;
an oligonucleotide probe capable of hybridizing with a nucleotide region of a 16S rRNA gene of gram-positive bacteria corresponding to positions 30-150 of SEQ ID NO 35, wherein the oligonucleotide probe consists of at least 10 contiguous nucleotides of a sequence selected from the group consisting of SEQ ID NOS: 25-27 or a complement of SEQ ID NOS: 25-27; and
an oligonucleotide probe capable of hybridizing with a nucleotide region of a 16S rRNA gene of gram-positive bacteria corresponding to positions 594-749 of SEQ ID NO 36, wherein the oligonucleotide probe consists of at least 10 contiguous nucleotides of a sequence selected from the group consisting of SEQ ID NOS: 28-30 or a complement of SEQ ID NOS: 28-30.

22. A method of discriminating gram-negative bacteria from gram-positive bacteria, the method comprising:
using genomic DNA in a sample as a template and a first oligonucleotide primer set specific for amplifying a target region specific to a gram-negative bacterium in a 16S rRNA gene and a second oligonucleotide primer set specific for amplifying a target region specific to a gram-positive bacterium in a 16S rRNA gene in an amplification reaction; and
detecting the gram-negative bacterium in the sample when the gram-negative bacterium-specific target sequence is amplified and detecting the gram-positive bacterium in the sample when the gram-positive bacterium-specific target sequence is amplified.


SEQUENCE LISTING (36 sequences)

SEQ ID NO: 1 - DNA, 20 bp, Artificial Sequence
  gramn1-F
gaggcagcagtggggaatwt

SEQ ID NO: 2 - DNA, 20 bp, Artificial Sequence
  primer (gramn1-R)
cccagtwawtccgattaacg

SEQ ID NO: 3 - DNA, 21 bp, Artificial Sequence
  primer (gramn2-F)
aacgatgcatacttgatgtgg

SEQ ID NO: 4 - DNA, 22 bp, Artificial Sequence
  primer (gramn2-R)
acgtgtgtcgccctggacataa

SEQ ID NO: 5 - DNA, 22 bp, Artificial Sequence
  primer (gramn3-F)
gtttgggatagccattggaaac

SEQ ID NO: 6 - DNA, 22 bp, Artificial Sequence
  primer (gramn3-R)
accgtcattatcttctctaaca

SEQ ID NO: 7 - DNA, 20 bp, Artificial Sequence
  primer (gramp1-F)
cgcattaagcactccgcctg

SEQ ID NO: 8 - DNA, 20 bp, Artificial Sequence
  primer (gramp1-R)
gagtgcccaactkaatgmtg

SEQ ID NO: 9 - DNA, 21 bp, Artificial Sequence
  primer (gramp2-F)
agtcgagcgaacagataagga

SEQ ID NO: 10 - DNA, 22 bp, Artificial Sequence
  primer (gramp2-R)
tatccggcattagccccggttt

SEQ ID NO: 11 - DNA, 22 bp, Artificial Sequence
  primer (gramp3-F)
aagtctggtgttaaaggcagct

SEQ ID NO: 12 - DNA, 22 bp, Artificial Sequence
  primer (gramp3-R)
agtaatggcctaagttttcgcc

SEQ ID NO: 13 - DNA, 20 bp, Artificial Sequence
  probe (gramn1-pr1)
ggcagcagtggggaatrttg

SEQ ID NO: 14 - DNA, 20 bp, Artificial Sequence
  probe (gramn1-pr2)
agccatgccgcgtgtrtgaa

SEQ ID NO: 15 - DNA, 20 bp, Artificial Sequence
  probe (gramn1-pr3)
atgccgcgtgtrtgaagaag

SEQ ID NO: 16 - DNA, 21 bp, Artificial Sequence
  probe (gramn2-pr1)
ttgtaaagcactttcgcctgg

SEQ ID NO: 17 - DNA, 21 bp, Artificial Sequence
  probe (gramn2-pr2)
gtaaagggcgtgtaggcggaa

SEQ ID NO: 18 - DNA, 21 bp, Artificial Sequence
  probe (gramn2-pr3)
atacctgacgctaaggcgcga

SEQ ID NO: 19 - DNA, 22 bp, Artificial Sequence
  probe (gramn3-pr1)
ttaataccagatactccctacg

SEQ ID NO: 20 - DNA, 22 bp, Artificial Sequence
  probe (gramn3-pr2)
atcagcctatgtcctatcagct

SEQ ID NO: 21 - DNA, 21 bp, Artificial Sequence
  probe (gramn3-pr3)
ccgcgtggaggatgaaggttt

SEQ ID NO: 22 - DNA, 20 bp, Artificial Sequence
  probe (gramp1-pr1)
gtttaattcgaagcaacgcg

SEQ ID NO: 23 - DNA, 20 bp, Artificial Sequence
  probe (gramp1-pr2)
ccgcaaggytgaaactcaaa

SEQ ID NO: 24 - DNA, 22 bp, Artificial Sequence
  probe (gramp1-pr3)
cgcaaggytgaaactcaaagga

SEQ ID NO: 25 - DNA, 21 bp, Artificial Sequence
  probe (gramp2-pr1)
agcatgctacggtgaatacgt

SEQ ID NO: 26 - DNA, 21 bp, Artificial Sequence
  probe (gramp2-pr2)
cgaagccggtggagtaaccat

SEQ ID NO: 27 - DNA, 22 bp, Artificial Sequence
  probe (gramp2-pr3)
agccggtggagtaaccattttg

SEQ ID NO: 28 - DNA, 22 bp, Artificial Sequence
  probe (gramp3-pr1)
ttaacagttgtatgcattggaa

SEQ ID NO: 29 - DNA, 22 bp, Artificial Sequence
  probe (gramp3-pr2)
agtgtggtagggagttttggaa

SEQ ID NO: 30 - DNA, 21 bp, Artificial Sequence
  probe (gramp3-pr3)
gaatttcatgtggagcggtga

SEQ ID NO: 31 - DNA, 1576 bp, Unknown
  gene (1)..(1576): consenus sequence of 16S rRNA genes of Gram-negative bacteria
gaattcactagwaattgaagagtttgatcmtggctcagattgaacgctggcggcaggcytaacacatgcaagtcgarcggtagcasgagddagcttgctytctgvtgacgagyggcggacgggtgagtaatgyhtrggaawctgcctgatrgwgggggataactackggaaacggtagctaataccgcatacgtcgcwasrssdaaagcgggggaccttcgggcctckygcyatyrgatgwgccyadrtsggattagctagttggtggggtaamggcyyaccaaggcgacgatcystagctggtctgagaggatgaycagccacactggractgagacacggyccagactcctacgggaggcagcagtggggaatattgsacaatgggcgcaagcctgatscagccatgccgcgtgtrtgaagaaggccttcgggttgtaaagyactttmagcggggaggaaggygwtawggttaatavcctyrmkcrattgacgttacccgcagaakaagcaccggctaactccgtgccagcagccgcggtaatacggagggtgcaagcgttaatcggaattactgggcgtaaagcgcrcgyaggcggtctvdttaagtcrgatgtgaaatccccgggctyaacctgggaactgcatyygawactggyargctagagtactkgtagaggggggtagaattccaggtgtagcggtgaaatgcgtagagatctggaggaataccgrtggcgaaggcrgccccctggmcwaakactgacgctsaggtgcgaaagcgtggggagcaaacaggattagataccctggtagtccacgccgtaaacgatgtckactwggmggttgkgsccttgaggcktrgctkscgsagctaacgcgwtaagthgaccgcctggggagtacggccgcaaggttaaaactcaaatgaattgacgggggcccgcacaagcggtggagcatgtggtttaattcgatgcaacgcgaagaaccttacctrstcttgacatmcwgagaatctttscagagatgmgaktrgtgccttcgggaactctgakacaggtgctgcatggctgtcgtcagctcgtgtygtgaratgttgggttaagtcccgcaacgagcgcaacccttdtccttwgttgccagcgagttcggycgggaactcwaaggagactgccrgtgayaaacyggaggaaggtggggatgacgtcaagtcatcatggcccttacgasyagggctacacacgtgctacaatggtcggatacaaagrgwwgcbamctcgcgagdgsaagckratctcataaagyvcgtcgtagtccggattggagtctgcaactcgactccatgaagtcggaatcgctagtaatcgyggatcagaatgccrcggtgaatacgttcccgggccttgtacacaccgcccgtcacaccatgggagtgggttgcammagaagtaggtagcytaaccttcrrgagggcgcttaccackktgtgattcatgactggggtgaagtcgtaacaaggtarccgtaggggaacctgcggytggatcacctccttamtmswattcccgcggcc

SEQ ID NO: 32 - DNA, 1557 bp, Unknown
  gene (1)..(1557): consensus sequence of 16S rRNA genes of Gram-negative bacteria
ttttctgagaatttgatcttagttcagattgaacgctggcggcgtggatgaggcatgcaagtcgaacggaataatgacttcggttgttrtttagtggcggaagggttagtartacatagataatctgccctcaacttgggrataacggttggaaacgatcgctaataccgaatgtrgtgtaattaggmatctaatatatattaaagaaggggatcttcggacctttcggttgaggaagagtttatgcgatatcagcttgttggtggggtaaaagcccaccaaggcgatgacgtctaggcggattgagagattgaccgccaacactgggactgagacactgcccagactcctaygggaggctgcagtcgagaatctttcgcaatggacgaaagtctgacgaagcgacgccgcgtgtgtgatgaaggccttagggttgtaaagcactttcgcctgggaataagagagrttggctaatatccaatygatttgagcgtaccrggtaaagaagcaccggctaactccgtgccagcagctgcggtaatacggagggtgctagcgttaatcggatttattgggcgtaaagggcgtgtaggcggaaaggaaagttagatgttaaattttggggctcaaccccaagtcagcatttaaaactatctttctagaggatagatggrgaaaagggaattccacgtgtagcggtgaaatgcgtagatatgtggaagaacaccagtggcgaaggcgcttttctaatttatacctgacgctaaggcgcgaaagcaaggggagcaaacaggattagataccctggtagtccttgccgtaaacgatgcatacttgatgtggatggtctcaaccccatccgtgtcggagctaacgtgttaagtatgccgcctgaggagtacactcgcaagggtgaaactcaaaagaattgacgggggcccgcacaagcagtggagcatgtggtttaattcgatgcaacgcgaagraccttacctggacttgacatgtatttgacaactgtagaaatacagctttccgcaaggacagatycacaggtgctgcatggctgtcgtcagctcgtgccgtgaggtgttgggttaagtcccgcaacgagcgcaacccttatcgttagttgccagcacttagggtgggaactctaacgagactgcctgggttaaccaggaggaaggcgaggatgacgtcaagtcagcatggcccttatgtccagggcgacacacgtgctacaatggttagtacagaaggtagcaasatcgtgagatggagcaaatcctaaaagctarccccagttcggattgtagtctgcaactcgactacatgaagtcggaattgctagtaatggcgtgtcagccataacgccgtgaatacgttcycgggccttgtacacaccgcccgtcacatcatgggagttggttttaccttaagtcgttgactcaacctatttataggagagaggcgcccaaggtgaggctgatgactgggatgaagtcgtaacaaggtagccctaccggaaggtggggctggatcacctccttttta

SEQ ID NO: 33 - DNA, 1765 bp, Unknown
  gene (1)..(1765): consensus sequence of 16S rRNA genes of Gram-negative bacteria
aagttcttcgtttggttggttggttaatactagaacacaaccccgttcttattcaataaagataaaacgagttcttgtgatagctaaaagctgttgttagaaataacaacagcttatcaaaaaacaaaagagctttaggacaaacacttttatggagagtttgatcctggctmrrrkkramssykggcggcgtgcctaatacatgcaagtcgaacgatgaagcttctagcttgctagarkgctgattagtggcgcacgggtgagtaacgcataggtyatgtgcctcttagtttgggatagccattggaaacgatgattaataccagatactccctacgggggaaagatttatcgctaagagatcagcctatgtcctatcagcttgttggtaaggtaatggcttaccaaggctatgacgggtatccggcctgagagggtgaacggacacactggaactgagacacggtccagactcctacgggaggcagcagtagggaatattgctcaatgggggaarccctgaagcagcaacgccgcgtggaggatgaaggttttaggattgtaaactccttttgttagagaagataatgacggtatctaacgaataagcaccggctaactccgtgccagcagccgcggtaatacgragggtgcaagcgttactcggaatcactgggcgtaaagagcgcgtaggcgggatagtcagtcaggtgtgaaatcctatggcttaaccatagaactgcatttgaaactactattctagagtgtgggagaggtaggtggaattcttggtgtaggggtaaaatccgtagagatcaagaggaatrctcatkgcgaaggcgacctgctggaacattactgacgctgattgcgcgaaagcgtggggagcaaacaggattagataccctggtagtccacgccctaaacgatggatgctagttgttggagggcttagtctytccagtaatgcagctaacgcattaagcatcccgcctggggagtacggtcgcaagattaaaactcaaaggaatagacggggacccgcacaagcggtggagcatgtggtttaattcgawkmtacacgaagaaccttacctaggcttgacattgagmgaatcygctagaaatagtggagtgtctrgcttgcyagaccttgaaaacaggtgctgcacggctgtcgtcagctcgtgtcgtgagatgttgggttaagtcccgcaacgagcgcaaccccctttcttagttgctaacaggtyatgctgagaactctaaggatactgcctccgtaaggaggaggaaggtggggacgacgtyaagtcatcatggcccttacgcctagggctacacacgtgctacaatggggtgcacaaagagaagcaatactgcgaagtggagccaatcttcaaaacacctctcagttcggattgtaggctgcaactcgcctgcatgaagctggaatcgctagtaatcgcaaatcagccatgttgcggtgaatacgttcccgggtcttgtactcaccgcccgtmacaccatggrasttgtgtttgccttaagtcaggatgctaaattrgctactgcccacggcacacacagcgactggggtgaagtcgtaacaaggtaaccgtagtgammcykssgktkgrwymmcyycytttctagagaaaagcttttaacattcgcttattaaaagccaaagagtattacctgacaaaagaacttcttgttgcttaggttttaaagattggggctttattcttttcttttttgt

SEQ ID NO: 34 - DNA, 1590 bp, Unknown
  gene (1)..(1590): consensus sequence of 16S rRNA genes of Gram-positive bacteria
atttdaatgagagtttgatcctggctcaggacgaacgctggcggcgtgcctaatacatgcaagtmgaacgmwcwgawgachggagcttgctacygtytgaatgagtkgcgaacgggtgagtaacrcgtrggtaacctrcctsatagmgggggataactattggaaacgrtagctaataccgcataabagtatkdaacwcgcatgkwtyhatrmttraaaggygswattgctvtcactatsagatggacctgcgttgtattagctagttggtraggtaacggctyaccaaggcracgatacatagccgacctgagagggtgatcggccacactgggactgagacacggcccagactcctacgggaggcagcagtagggaatcttcggcaatgggsgraascctgaccgagcaacgccgcgtgagtgaagaaggttttcggatcgtaaagctctgttgttagrgaagaacgawgtgtgagagtrgaaagtycacacmtgtgacggtahctaaccagaaagssacggctaactacgtgccagcagccgcggtaatacgtaggtsscragcgttgtccggatttattgggcgtaaagcgagcgcaggcggtttkwtaagtctgawgtkaaagschgyggctyaaccrtdgwrsgcwttggaaactgkkagaacttgagtgcagaagrggagagtggaattccatgtgtagcggtgaaatgcgtagatatatggaggaacaccrgtggcgaargcggctctctggtctgtaactgacgctgaggctcgaaagcgtggggagcaaacaggattagataccctggtagtccacgccgtaaacgatgagtgctargtgttaggssstttccgssscttagtgcygcagctaacgcattaagcactccgcctggggagtacgaccgcaaggttgaaactcaaaggaattgacgggggcccgcacaagcggtggagcatgtggtttaattcgaagcaacgcgaagaaccttaccaggtcttgacatccckhtgaccrctctagagatagagmtttyccttcgggrcabmargtgacaggtggtgcatggttgtcgtcagctcgtgtcgtgagatgttgggttaagtcccgcaacgagcgcaacccytattgttagttgccatcattmagttgggcactctagcgagactgccggtrayaaaccggaggaaggtggggatgacgtcaaatcatcatgccccttatgacctgggctacacacgtgctacaatgghtrgtacaacgagtcgcragccggtgacggcaagctaatctcttaaagcyadtctcagttcggattgtaggctgcaactcgcctacatgaagycggaatcgctagtaatcgcggatcagcacgccgcggtgaatacgttcccgggccttgtacacaccgcccgtcacaccacgagagtttgtaacacccgaagtcggtgaggtaaccttttaggagccagccgcctaaggtgggatagatgattggggtgaagtcgtaacaaggtagccgtatcggaaggtgcggctggatcacctcctttctaagctkgdatccccggtaccgagct

SEQ ID NO: 35 - DNA, 1213 bp, Unknown
  gene (1)..(1213): consensus sequence of 16S rRNA genes of Gram-positive bacteria
gaacgctggcggcgtgcctaatacatgcaagtcgagcgaacagataaggagcttgctcctttgacgttagcggcggacgggtgagtaacacgtgggtaacctacctataagactggaataactccgggaaaccggggctaatgccggataacatdtagaaccgcatggttctahagtgaaagatggttttgctatcacttatagatggacccgcgccgtattagctagttggtaaggtaacggcttaccaaggcgacgatacgtagccgacctgagagggtgatcggccacactggaactgagacacggtccagactcctacgggaggcagcagtagggaatcttccgcaatgggcgaaagcctgacggagcaacgccgcgtgagtgatgaaggtcttcggatcgtaaaactctgttattagggaagaacaaatgtgtaagtaactgtgcacatcttgacggtacctaatcagaaagccacggctaactacgtgccagcagccgcggtaatacgtaggtggcaagcgttatccggaattattgggcgtaaagcgcgcgtaggcggtttyttaagtctgatgtgaaagcccacggctcaaccgtggagggtcattggaaactggrgaaacttgagtgcaraagagraaagtggaattccatgtgtagcggtgaaatgcscararatatggaggaacaccagtggcgaaggygacwkkctggtgcatggtwryygwcmgctsrtgtcgyraraygttgggktaagtcmmrcarcgakmrmwacccttgracttagttgccaycmytmasttgggcactctaagttgactgccggtgacaaaccggaggaaggtggggatgacgtcaaatcatcatgccccttatgatttgggctacacacgtgctacaatggacaatacaaagggcagcgaaaccgcgaggtcaagcaaatcccataaagttgttctcagttcggattgtagtctgcaactcgactacatgaagctggaatcgctagtaatcgtagatcagcatgctacggtgaatacgttcccgggtcttgtacacaccgcccgtcacaccacgagagtttgtaacacccgaagccggtggagtaaccattttggagctagccgtcgaaggtgggacaaatgattggggtgaagtcgtaacaaggtagccgtatcggaaggtgcggctggatcacctcctttct

SEQ ID NO: 36 - DNA, 1518 bp, Unknown
  gene (1)..(1518): consensus sequence of 16S rRNA genes of Gram-positive bacteria
atttttctgagagtttgatcctggctcaggattaacgctggcggcatgcctaatacatgcaagtcgatcgaaagtagtaatactttagaggcgaacgggtgagtaacacgtatccaatctaccttataatgggggataactagttgaaagactagctaataccgcataagaactttggttcgcatgaatcaaagttgaaaggacctgcaagggttcgttatttgatgagggtgcgccatatcagctagttggtggggtaacggcctaccaaggcaatgacgtgtagctatgctgagaagtagaatagccacaatgggactgagacacggcccatactcctacgggaggcagcagtagggaatttttcacaatgagcgaaagcttgatggagcaatgccgcgtgaacgatgaaggtctttaagattgtaaagttcttttatttgggaagaatgactttagcaggtaatggctagagtttgactgtaccattttgaataagtgacgactaactatgtgccagcagtcgcggtaatacataggtcgcaagcgttatccggatttattgggcgtaaagcaagcgcaggcggattgaaaagtctggtgttaaaggcagctgcttaacagttgtatgcattggaaactattaatctagagtgtggtagggagttttggaatttcatgtggagcggtgaaatgcgtagatatatgaaggaacaccagtggcgaaggcgaaaacttaggccattactgacgcttaggcttgaaagtgtggggagcaaataggattagataccctagtagtccacaccgtaaacgatagatactagctgtcggggcgatcccctcggtagtgaagttaacacattaagtatctcgcctgggtagtacattcgcaagaatgaaactcaaacggaattgacggggacccgcacaagtggtggagcatgttgcttaattcgacggtacacgaaaaaccttacctagacttgacatccttggcaaagttatggaaacataatggaggttaaccgagtgacaggtggtgcatggttgtcgtcagctcgtgtcgtgagatgttgggttaagtcccgcaacgagcgcaacccttatcgttagttacattgtctagcgagactgctaatgcaaattggaggaaggaagggatgacgtcaaatcatcatgccccttatgtctagggctgcaaacgtgctacaatggccaatacaaacagtcgccagcttgtaaaagtgagcaaatctgtaaagttggtctcagttcggattgagggctgcaattcgtcctcatgaagtcggaatcactagtaatcgcgaatcagctatgtcgcggtgaatacgttctcgggtcttgtacacaccgcccgtcaaactatgaaagctggtaatatttaaaaacgtgttgctaaccattaggaagcgcatgtcaaggatagcaccggtgattggagttaagtcgtaacaaggtacccctacgagaacgtgggggtggatcacctccttt